(19) United States
(12) Patent Application Publication
(10) Pub. No.: US 2009/0259217 A1
(43) Pub. Date: Oct. 15, 2009

(54) Methods and systems associated with delivery of one or more agents to an individual

(75) Inventors: Roderick A. Hyde, Redmond, WA (US); Muriel Y. Ishikawa, Livermore, CA (US); Lowell L. Wood, JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 12/157,159

(22) Filed: Jun. 5, 2008

Related U.S. Application Data
(63) Continuation-in-part of application No. 12/082,402, filed on Apr. 9, 2008.
Application No. 12/082,402 is a continuation-in-part of application No. 12/082,403, filed on Apr. 9, 2008.
Application No. 12/082,403 is a continuation-in-part of application No. 12/082,404, filed on Apr. 9, 2008.

(51) Int. Cl. A61M 5/168

(52) U.S. Cl. 604/891.1; 604/890.1; 604/66

(57) ABSTRACT

The present disclosure relates to methods and systems for administration of one or more agents.

### CROSS-REFERENCE TO RELATED APPLICATIONS

The present application is related to and claims the benefit of the earliest available effective filing date(s) from the following listed application(s) (the “Related Applications”) (e.g. claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. UNKNOWN, entitled AGENT DELIVERY DEVICE, naming Roderick A. Hyde, Muriel Y. Ishikawa, and Lowell L. Wood, Jr. as inventors, filed 9 Apr. 2008, which is currently co-pending, or is an application of which a currently co-pending application is entitled to the benefit of the filing date.

For purposes of the USPTO extra-statutory requirements, the present application constitutes a continuation-in-part of U.S. patent application Ser. No. UNKNOWN, entitled SENSORS, naming Roderick A. Hyde, Muriel Y. Ishikawa, and Lowell L. Wood, Jr. as inventors, filed 9 Apr. 2008, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

The present disclosure relates to methods and systems for administration of one or more agents.

### SUMMARY

In one aspect, a method includes but is not limited to receiving one or more signals that include information related to one or more agents from one or more sensors that are implanted within an individual and administering one or more agents to the individual with one or more implanted agent delivery devices in response to the one or more signals. In addition to the foregoing, other method aspects are described in the claims, drawings, and text forming a part of the present disclosure.

In one aspect, a system includes but is not limited to circuitry for receiving one or more signals that include information related to one or more agents from one or more sensors that are implanted within an individual and circuitry for administering one or more agents to the individual with one or more agent delivery devices that are implanted within the individual and responsive to the circuitry for receiving the one or more signals. In addition to the foregoing, other system aspects are described in the claims, drawings, and text forming a part of the present disclosure.

In one aspect, a system includes but is not limited to means for receiving one or more signals that include information related to one or more agents from one or more sensors that are implanted within an individual and means for administering one or more agents to the individual with one or more agent delivery devices that are implanted within the individual and responsive to the means for receiving the one or more signals. In addition to the foregoing, other system aspects are described in the claims, drawings, and text forming a part of the present disclosure.

In one aspect, a system includes but is not limited to a signal-bearing medium bearing one or more instructions for receiving one or more signals that include information related to one or more agents from one or more sensors that are implanted within an individual and one or more instructions for administering one or more agents to the individual with one or more implanted agent delivery devices in response to the one or more signals. In addition to the foregoing, other system aspects are described in the claims, drawings, and text forming a part of the present disclosure.

In one or more various aspects, means include but are not limited to circuitry and/or programming for effecting the herein referenced functional aspects; the circuitry and/or programming can be virtually any combination of hardware, software, and/or firmware configured to effect the herein referenced functional aspects depending upon the design choices of the system designer. In addition to the foregoing, other system aspects means are described in the claims, drawings, and/or text forming a part of the present disclosure.

In one or more various aspects, related systems include but are not limited to circuitry and/or programming for effecting the herein-referenced method aspects; the circuitry and/or programming can be virtually an), combination of hardware, software, and/or firmware configured to effect the herein referenced method aspects depending upon the design choices of the system designer. In addition to the foregoing, other system aspects are described in the claims, drawings, and/or text forming a part of the present application.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

FIG. 1 illustrates an example system 100 in which embodiments may be implemented.

FIG. 2 illustrates an operational flow representing example operations related to methods and systems associated with the example system 100.

FIG. 3 illustrates alternate embodiments of the example operational flow of FIG. 2.

FIG. 4 illustrates alternate embodiments of the example operational flow of FIG. 2.

FIG. 5 illustrates alternate embodiments of the example operational flow of FIG. 2.

FIG. 6 illustrates alternate embodiments of the example operational flow of FIG. 2.

FIG. 7 illustrates alternate embodiments of the example operational flow of FIG. 2.

FIG. 8 illustrates alternate embodiments of the example operational flow of FIG. 2.

FIG. 9 illustrates alternate embodiments of the example operational flow of FIG. 2.

FIG. 10 illustrates alternate embodiments of the example operational flow of FIG. 2.

FIG. 11 illustrates alternate embodiments of the example operational flow of FIG. 2.

FIG. 12 illustrates alternate embodiments of the example operational flow of FIG. 2.

FIG. 13 illustrates alternate embodiments of the example operational flow of FIG. 2.

FIG. 14 illustrates alternate embodiments of the example operational flow of FIG. 2.

FIG. 15 illustrates alternate embodiments of the example operational flow of FIG. 2.

FIG. 16 illustrates alternate embodiments of the example operational flow, of FIG. 2.

FIG. 17 illustrates alternate embodiments of the example operational flow of FIG. 2.

FIG. 18 illustrates a partial view of a system 1800 that includes a computer program for executing a computer process on a computing device.

FIG. 19A illustrates an embodiment of an agent delivery device.

FIG. 19B illustrates an embodiment of an agent delivery device.

FIG. 20A illustrates an embodiment of an agent delivery device.

FIG. 20B illustrates an embodiment of an agent delivery device.

FIG. 211A illustrates an embodiment of an agent delivery device.

FIG. 21B illustrates an embodiment of an agent delivery device.

FIG. 29A illustrates an embodiment of an agent delivery device.

FIG. 29B illustrates an embodiment of an agent delivery device.

FIG. 23 illustrates an embodiment of an agent delivery device.

FIG. 24 illustrates an embodiment of an agent delivery device.

FIG. 25A illustrates an embodiment of an agent delivery device.

FIG. 25B illustrates an embodiment of an agent delivery device.

FIG. 26A illustrates an embodiment of an agent delivery device.

FIG. 26B illustrates an embodiment of an agent delivery device.

FIG. 27A illustrates an embodiment of an agent delivery device.

FIG. 27B illustrates an embodiment of an agent delivery device.

FIG. 28A illustrates an embodiment of an agent delivery device.

FIG. 28B illustrates an embodiment of an agent delivery device.

FIG. 29A illustrates an embodiment of an agent delivery device.

FIG. 29B illustrates an embodiment of an agent delivery device.

FIG. 30A illustrates an embodiment of a sensor.

FIG. 30B illustrates an embodiment of a sensor.

FIG. 30C illustrates an embodiment of a sensor.

FIG. 31A illustrates an embodiment of a sensor.

FIG. 31B illustrates an embodiment of a sensor.

FIG. 31C illustrates an embodiment of a sensor.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

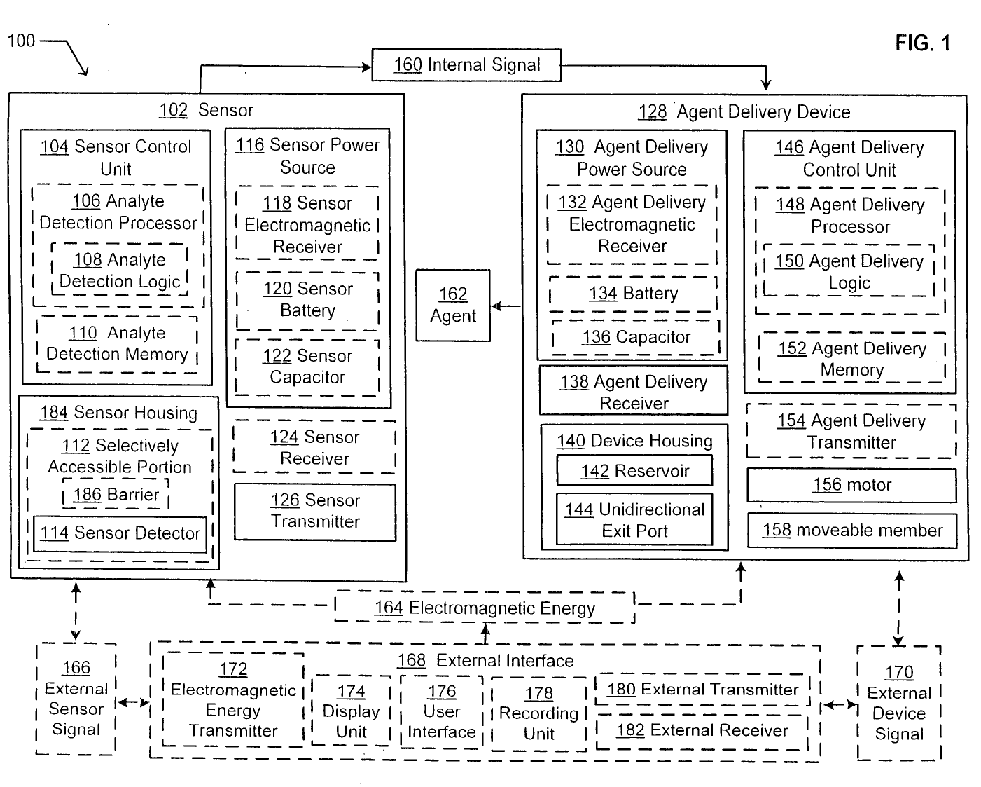

FIG. 1 illustrates an example system 100 in which embodiments may be implemented. In some embodiments, system 100 may include one or more sensors 102. In some embodiments, system 100 may include one or more agent delivery devices 128. In some embodiments, system 100 may include one or more external interfaces 168. In some embodiments, one or more sensors 102 may be configured to transmit one or more internal signals 160. In some embodiments, one or more agent delivery devices 128 may be configured to receive one or more internal signals 160. In some embodiments, one or more agent delivery devices 128 may be configured to administer one or more agents 162. In some embodiments, one or more external interfaces 168 may be configured to transmit electromagnetic energy 164. In some embodiments, one or more external interfaces 168 may be configured to transmit one or more external sensor signals 166. In some embodiments, one or more external interfaces 168 may be configured to transmit one or more external device signals 170.

In some embodiments, one or more agent delivery devices 128 may include one or more agent delivery receivers 138 that may be configured to receive one or more internal signals 160. In some embodiments, one or more agent delivery devices 128 may include one or more agent delivery receivers 138 that may be configured to receive one or more external device signals 170. In some embodiments, one or more agent delivery devices 128 may include one or more agent delivery transmitters 154 that may be configured to transmit one or more external device signals 170. In some embodiments, one or more agent delivery devices 128 may include one or more agent delivery power sources 130. In some embodiments, one or more agent delivery devices 128 may include one or more agent delivery electromagnetic receivers 132 that may be configured to receive electromagnetic energy 164. In some embodiments, one or more agent delivery devices 128 may include one or more batteries 134. In some embodiments, one or more agent delivery devices 128 may include one or more capacitors 136. In some embodiments, one or more agent delivery devices 128 may include one or more housings 140. In some embodiments, one or more agent delivery devices 198 may include one or more reservoirs 142. In some embodiments, one or more agent delivery devices 128 may include one or more unidirectional exit ports 144. In some embodiments, one or more agent delivery devices 128 may include one or more agent delivery control units 146. In some embodiments, one or more agent delivery devices 128 may include one or more agent delivery processors 148. In some embodiments, one or more agent delivery devices 128 may include agent delivery logic 150. In some embodiments, one or more agent delivery devices 128 may include agent delivery memory 152. In some embodiments, one or more agent delivery devices 128 may include one or more motors 156. In some embodiments, one or more agent delivery devices 128 may include one or more moveable members 158.

In some embodiments, one or more sensors 102 may include one or more sensor transmitters 126 that may be configured to transmit one or more internal signals 160. In some embodiments, one or more sensors 102 may include one or more sensor transmitters 126 that may be configured to transmit one or more external sensor signals 166. In some embodiments, one or more sensors 102 may include one or more sensor receivers 124 that may be configured to receive one or more external sensor signals 166. In some embodiments, one or more sensors 102 may include one or more sensor power sources 116. In some embodiments, one or more sensors 102 may include one or more sensor electromagnetic receivers 118 that may be configured to receive electromagnetic energy 164. In some embodiments, one or more sensors 102 may include one or more sensor batteries 120. In some embodiments, one or more sensors 102 may include one or more sensor capacitors 129. In some embodiments, one or more sensors 102 may include one or more sensor control units 104. In some embodiments, one or more sensors 102 may include one or more analyte detection processors 106. In some embodiments, one or more sensors 102 may include analyte detection logic 108. In some embodiments, one or more sensors 102 may include analyte detection memory 110. In some embodiments, one or more sensors 102 may include one or more selectively accessible portions 112. In some embodiments, one or more sensors 102 may include one or more detectors 114.

In some embodiments, system 100 may include one or more external interfaces 168 that include one or more external receivers 182 that are configured to receive one or more external sensor signals 166. In some embodiments, system 100 may include one or more external interfaces 168 that include one or more external receivers 182 that are configured to receive one or more external device signals 170. In some embodiments, system 100 may include one or more external interfaces 168 that include one or more external transmitters 180 that are configured to transmit one or more external sensor signals 166. In some embodiments, system 100 may include one or more external interfaces 168 that include one or more external transmitters 180 that are configured to transmit one or more external device signals 170. In some embodiments, system 100 may include one or more external interfaces 168 that include one or more electromagnetic energy transmitters 172 that are configured to transmit electromagnetic energy 164. In some embodiments, system 100 may include one or more external interfaces 168 that include one or more user interfaces 176. In some embodiments, system 100 may include one or more external interfaces 168 that include one or more display units 174. In some embodiments, system 100 may include one or more external interfaces 168 that include one or more recording units 178.

### Sensor

System 100 may include one or more sensors 102. In some embodiments, one or more sensors 102 may be configured for implantation within an individual (e.g., U.S. Pat. Nos. 7,110,803 and 7,044,911). Sensors 102 may be configured for implantation at numerous positions within an individual. For example, in some embodiments, one or more sensors 102 may be configured for implantation into the vasculature of an individual (e.g., U.S. Pat. Nos. 7,181,261; 7,025,734; and 7,236,821).

A sensor 102 may be operably associated with one or more sensor control units 104. In some embodiments, the one or more sensor control units 104 may serve to regulate the activity of one or more sensors 102. For example, in some embodiments, one or more sensor control units 104 may regulate one or more times when one or more sensors 109 detect one or more analytes. In some embodiments, one or more sensor control units 104 may regulate one or more time periods when one or more sensors 102 detect one or more analytes. In some embodiments, one or more sensor control units 104 may regulate what analytes are detected by one or more sensors 102. In some embodiments, one or more sensor control units 104 may regulate unmasking of one or more selectively accessible portions 112 of one or more sensors 102. For example, in some embodiments, one or more sensor control units 104 may regulate unmasking of selectively accessible portions 112 of one or more sensors 102 to expose one or more sensor detectors 114 at one or more times. Accordingly, in some embodiments, one or more sensor control units 104 may regulate which sensor detectors 114 are available for detection of one or more analytes and when the one or more detectors are made available. In some embodiments, one or more sensor control units 104 may be operably coupled to one or more analyte detection processors 106. In some embodiments, one or more sensors 102 may include a processing unit that is configured to process information received from one or more sensor detectors 114. For example, in some embodiments, one or more analyte detection processors 106 may be configured to calculate the concentration of one or more detected analytes. In some embodiments, one or more analyte detection processors 106 may be configured to determine changes in the concentration of one or more detected analytes relative to time. In some embodiments, one or more analyte detection processors 106 may be configured to determine changes in the concentration of one or more detected analytes relative to one or more amounts of agent 162 that are administered to an individual. In some embodiments, one or more analyte detection processors 106 may be configured to prepare one or more instructions for one or more agent delivery devices 128. For example, in some embodiments, one or more analyte detection processors 106 may instruct one or more agent delivery devices 128 to administer one or more amounts of one or more agents 162. In some embodiments, one or more analyte detection processors 106 may instruct one or more agent delivery devices 128 to administer one or more agents 162 at one or more times. In some embodiments, one or more analyte detection processors 106 may instruct one or snore agent delivery devices 128 to administer one or more amounts of one or more agents 162 at one or more times. In some embodiments, one or more analyte detection processors 106 may include analyte detection logic 108. For example, in some embodiments, one or more analyte detection processors 106 may include anal rte detection logic 108 that is programmed to compensate for background occurring during detection of one or more analytes. In some embodiments, analyte detection logic 108 may be configured to process information obtained during detection of one or more analytes to account for the personal characteristics of the individual into which the sensor 102 is implanted. For example, in some embodiments, analyte detection logic 108 may be configured to determine the amount of one or more agents 162 to be administered to an individual to maintain the concentration of the one or more agents 162 at one or more setpoints within the individual. In some embodiments, analyte detection logic 108 may be configured to determine the amount of one or more agents 162 to be administered to an individual to maintain the concentration of the one or more agents 162 within one or more concentration ranges within the individual. In some embodiments, a sensor control unit 104 may include analyte detection memory 110. For example, in some embodiments, one or more sensors 102 may save information associated with the identity of one or more detected analytes, the identity of one or more undetected analytes, the concentration of one or more analytes, changes in the concentration of one or more analytes, or substantially any combination thereof. Numerous types of memory may be used for analyte detection memory 110. Examples of memory include, but are not limited to, flash memory, random access memory, read-only memory, and the like.

In some embodiments, a sensor 102 may include one or more sensor housings 184. In some embodiments, one or more sensor housings 184 may be operably coupled with one or more sensor detectors 114. In some embodiments, one or more sensor housings 184 may include one or more selectively accessible portions 112. In some embodiments, one or more sensor housings 184 may include one or more selectively accessible portions 112 that enclose one or more sensor detectors 114. In some embodiments, one or more selectively accessible portions 112 may include one or more structures that modulate access to the one or more selectively accessible portions 112 of the sensor housing 184. For example, in some embodiments, one or more selectively accessible portions 192 may be covered with a gold sacrificial layer that may be r emoted through electrochemical dissolution with a constant DC current (e.g., 35 mA/cm²) (Pan et al., Proceedings of the 26^{th} Annual International Conference of the IEEE EMBS, San Francisco, Calif., USA, Sep. 1-5, 2004). In some embodiments, one or more selectively accessible portions 112 may be covered with a shape-memory polymer that may be activated to unsequester the one or more selectively accessible portions 112 (e.g., U.S. Pat. No. 6,454,759). In some embodiments, the one or more selectively accessible portions 112 may sequester one or more sensor detectors 114 that may be selectively unsequestered. Numerous types of sensor detectors 114 may be associated with one or more sensors 102. Examples of such sensor detectors 114 include, but are not limited to, electrodes, surface plasmon resonance detectors 114, microelectromechanical systems detectors 114, microcantilever detectors 114, nitric oxide detectors 114, osmotic detectors 114, relativity-based detectors 114, chemical detectors 114, pressure detectors 114, electrochemical detectors 114, piezoelectric detectors 114, pH detectors 114, hydrogel detectors 114, enzymatic detectors 114, ball integrated circuit detectors 114, affinity viscosimetric detectors 114, blood pressure detectors 114; metal detectors 114, and the like (e.g., U.S. Pat. Nos. 7,169,289; 6,280,604; 5,603,820; 5,582,170; 6,287,459; 7,291,503; 6,764,446; 7,168,294; 6,823,717; 7,205,701; 6,268,161; 4,703,756; 6,965,791; 6,546,268; 6,210,326; 6,514,689; 6,934,973; 6,442,413; Tu et al., Electroanalysis, 11:70-74 (1999), Malinski et al., Molecular Mechanisms of Metal Toxicity and Carcinogenicity, Environmental Health Perspectives 102, Supplement 3, September 1994).

In some embodiments, a sensor 102 may include one or more sensor power sources 116. In some embodiments, a sensor 102 may be operably coupled to one or more sensor batteries 120. In some embodiments, a sensor battery 120 may include a thin-film fuel cell for providing electrical power. In some embodiments, the fuel cell may be of a solid oxide type (SOFC), a solid polymer type (SPFC), a proton exchange membrane type (PEFC), and/or substantially any combination thereof. Methods to fabricate such thin-film fuel cells are known and have been described (e.g., U.S. Pat. No. 7,189,471). In some embodiments, one or more sensor batteries 120 may include one or more storage films that are configured for energy storage and energy conversion. Methods to fabricate such storage films are known and have been described (e.g., U.S. Pat. No. 7,938,628). In some embodiments, a sensor battery 120 may be a biobased battery (e.g., U.S. Pat. No. 6,994,934). In some embodiments, one or more sensor batteries 120 may be thin film batteries. Methods to fabricate thin-film batteries are known and have been described (e.g., U.S. Pat. Nos. 7,194,801; 7,144,655; 6,818,356). In some embodiments, one or more sensor electromagnetic receivers 118 may be used to electromagnetically couple power to energize one or more sensors 102 from an external power source. Methods to construct electromagnetic receivers have been described (e.g., U.S. Pat. No. 5,571,152). Briefly, in some embodiments, one or more electromagnetic receivers may be associated with one or more rectifier chips. The one or more sensor electromagnetic receivers 118 may include one or more cores about which are wrapped an electrical conductor. In some embodiments, cores may comprise a material, such as a ferrite material, due to its relatively high magnetic permeability and low magnetic hysteresis. However, other materials can be used for this purpose. In some embodiments, a sensor 102 may be operably coupled to one or more sensor capacitors 122. In some embodiments, one or more sensor electromagnetic receivers 118 may be operably coupled to one or more batteries. In some embodiments, one or more sensor electromagnetic receivers 118 may be operably coupled to one or more sensor capacitors 122. Accordingly, in some embodiments, one or more sensors 102 mail be configured such that they are operably coupled to a rechargeable power source.

The system 100 may include one or more sensor transmitters 126. Numerous types of sensor transmitters 126 may be used in association with system 100. Examples of such sensor transmitters 126 include, but are not limited to, transmitters that transmit one or more acoustic signals, optical signals, radio signals, wireless signals, hardwired signals, infrared signals, ultrasonic signals, and the like (e.g., U.S. Pat. Nos. RE39,785; 7,260,768; 7,260,764; 7,260,402; 7,257,327; 7,215,887; 7,218,900). In some embodiments, one or more sensor transmitters 196 may transmit one or more signals that are encrypted. Numerous types of transmitters are known and have been described (e.g., U.S. Pat. Nos. and Published U.S. Patent Applications: 7,236,595; 7,260,155; 7,227,956; US2006/0280307).

The system 100 may include one or more sensor receivers 124. Numerous types of sensor receivers 194 may be used in association with system 100. Examples of such sensor receivers 124 include, but are not limited to, receivers that receive one or more acoustic signals, optical signals, radio signals, wireless signals, hardwired signals, infrared signals, ultrasonic signals, and the like. Such receivers are known and have been described (e.g., U.S. Pat. Nos. RE39,785; 7,218,900; 7,254,160; 7,245,894; 7,206,605).

### Agent Delivery Device

The system 100 may include one or more agent delivery devices 128. In some embodiments, an agent delivery device 128 may be configured for implantation within an individual.

In some embodiments, an agent delivery device 128 may include one or more agent delivery power sources 130. In some embodiments, an agent delivery device 128 may be operably coupled to one or more batteries 134. In some embodiments, a battery 134 may include a thin-film fuel cell for providing electrical power. In some embodiments, the fuel cell may be of a solid oxide type (SOFC), a solid polymer type (SPFC), a proton exchange membrane type (PEMFC), and/or substantially any combination thereof. Methods to fabricate such thin-film fuel cells are known and have been described (e.g., U.S. Pat. No. 7,189,471). In some embodiments, one or more batteries 134 may include one or more storage films that are configured for energy storage and energy conversion. Methods to fabricate such storage films are known and have been described (e.g., U.S. Pat. No. 7,238,628). In some embodiments, a battery 134 may be a biobased battery 134 (e.g., U.S. Pat. No. 6,994,934). In some embodiments, one or more batteries 134 may be thin film batteries 134. Methods to fabricate thin-film batteries 134 are known and have been described (e.g., U.S. Pat. Nos. 7,194,801; 7,144,655; 6,818,356). In some embodiments, one or more agent delivery electromagnetic receivers 132 may be used to electromagnetically couple power to energize one or more agent delivery devices 128 from an external power source. Methods to construct electromagnetic receivers have been described (e.g., U.S. Pat. No. 5,571,152). Briefly, in some embodiments, one or more electromagnetic receivers may be associated with one or more rectifier chips. The one or more agent delivery electromagnetic receivers 132 may include one or more cores about which are wrapped an electrical conductor. In some embodiments, cores may comprise a material, such as a ferrite material, due to its relatively high magnetic permeability, and low magnetic hysteresis. However, other materials can be used for this purpose. In some embodiments, an agent delivery device 198 may be operably coupled to one or more capacitors 136. In some embodiments, one or more agent delivery electromagnetic receivers 132 mall be operably coupled to one or more batteries 134. In some embodiments, one or more agent delivery electromagnetic receivers 139 may be operably coupled to one or more capacitors 136. Accordingly, in some embodiments, one or more agent delivery devices 128 may be configured such that they are operably coupled to a rechargeable power source.

An agent delivery device 128 may be operably associated with one or more agent delivery control units 146. In some embodiments, the one or more agent delivery control units 146 may serve to regulate the activity of one or more agent delivery devices 128. For example, in some embodiments, one or more agent delivery control units 146 may regulate one or more times when one or more agent delivery devices 128 administer one or more agents 162. In some embodiments, one or more agent delivery control units 146 may regulate one or more time periods when one or more agent delivery devices 128 administer one or more agents 162. In some embodiments, one or more agent delivery control units 146 may regulate what agents 162 are administered by one or more agent delivery devices 128. In some embodiments, one or more agent delivery control units 146 may regulate the operation of one or more motors 156 associated with one or more agent delivery devices 128. For example, in some embodiments, one or more agent delivery control units 146 may regulate the duration of operation of one or more motors 156. In some embodiments, one or more agent delivery control units 146 may regulate the time when one or more motors 156 are operated. In some embodiments, one or more agent delivery control units 146 may regulate the frequency with which one or more motors 156 are operated. In some embodiments, one or more agent delivery control units 146 may be operably coupled to one or more agent delivery processors 148. In some embodiments, one or more agent delivery devices 128 may include a processing unit that is configured to process information received from one or more sensors 102. For example, in some embodiments, one or more agent delivery processors 148 may be configured to calculate the concentration of one or more detected analytes. In some embodiments, one or more agent delivered processors 148 may be configured to determine changes in the concentration of one or more detected analytes relative to time. In some embodiments, one or more agent delivery processors 148 may be configured to determine changes in the concentration of one or more detected analytes relative to one or more amounts of agent 162 that are administered to an individual. In some embodiments, one or more agent delivery processors 148 may be configured to regulate one or more motors 156 that are operably coupled to the agent delivery device 128. For example, in some embodiments, one or more agent delivery processors 148 may facilitate operation of one or more motors 156 to administer one or more amounts of one or more agents 162. In some embodiments, one or more agent delivery processors 148 may facilitate operation of one or more motors 156 to administer one or more agents 162 at one or more times. In some embodiments, one or more agent delivery processors 148 may facilitate operation of one or more motors 156 to administer one or more amounts of one or more agents 162 at one or more times. In some embodiments, one or more agent delivery processors 148 may include agent delivery logic 150. For example, in some embodiments, one or more agent delivery processors 148 may include agent delivery logic 150 that is programmed to facilitate administration of one or more agents 162 to an individual. In some embodiments, one or more agent delivery processors 148 may include agent delivery logic 150 that is programmed to facilitate administration of one or more agents 162 to an individual such that the concentration of the one or more agents 162 is substantially maintained at a setpoint. In some embodiments, one or more agent delivery processors 148 may include agent delivery logic 150 that is programmed to facilitate administration of one or more agents 162 to an individual such that the concentration of the one or more agents 162 is substantially maintained within a range of concentrations. In some embodiments, one or more agent delivery processors 148 may include agent delivery logic 150 that is programmed to facilitate administration of one or more agents 162 to an individual with regard to characteristics of the individual. For example, in some embodiments, agent delivery logic 150 may account for the size of an individual to facilitate administration of one or more agents 162 to an individual. In some embodiments, an agent delivery, control unit 146 may, include agent delivery memory 159. For example, in some embodiments, one or more agent delivery devices 128 may save information associated with the identity of one or more administered agents 162, the concentration of one or more administered agents 162, changes in the concentration of one or more agents 162, or substantially any combination thereof. Numerous types of memory may be used for agent delivery memory 152. Examples of memory include, but are not limited to, flash memory, random access memory, read-only memory, and the like.

An agent delivery device 128 may include one or more agent delivery transmitters 154. Numerous types of agent delivery transmitters 154 may be used in association with system 100. Examples of such agent delivery transmitters 154 include, but are not limited to, transmitters that transmit one or more acoustic signals, optical signals, radio signals, wireless signals, hardwired signals, infrared signals, ultrasonic signals, and the like (e.g., U.S. Pat. Nos. RE39,785; 7,260,768; 7,960,764; 7,260,402; 7,257,327; 7,215,887; 7,218,900; herein incorporated by reference). In some embodiments, one or more agent delivery transmitters 154 may transmit one or more signals that are encrypted. Numerous types of transmitters are known and have been described (e.g., U.S. Pat. Nos. and Published U.S. Pat. Nos. 7,236,595; 7,260,155; 7,297,956; US2006/0280307; herein incorporated by reference).

An agent delivery device 128 may include one or more agent delivery receivers 1318. Numerous types of agent delivery receivers 138 may, be used in association with system 100. Examples of such agent delivery receivers 138 include, but are not limited to, receivers that receive one or more acoustic signals, optical signals, radio signals, wireless signals, hardwired signals, infrared signals, ultrasonic signals, and the like. Such receivers are known and have been described (e.g., U.S. Pat. Nos. RE39,785; 7,218,900; 7,254,160; 7,245,894; 7,206,605; herein incorporated by reference).

An agent delivery device 128 may include one or more device housings 140. In some embodiments, one or more device housings 140 may include one or more reservoirs 142. In some embodiments, one or more device housings 140 may be operably coupled with one or more unidirectional exit ports 144. In some embodiments, one or more device housings 140 may be operably coupled with one or more motors 156. In some embodiments, one or more device housings 140 may be operably coupled with one or more moveable members 158. For example, in some embodiments, a device housing 140 may be configured as a tube with a unidirectional exit port 144 operably coupled to a distal end of the tube. In some embodiments, such a device housing 140 may be configured to accept a moveable member 158 that is configured to slide within the interior of the device housing tube from a proximal end of the tube to the distal end of the tube. In some embodiments, the moveable member 158 may be operably associated with one or more motors 156 that are configured to translocate the moveable member 158. In some embodiments, the space within the tube between the moveable member 158 and the unidirectional exit port 144 may be configured as a reservoir 142 that may include one or more agents 162. Accordingly, movement of the moveable member 158 from the proximal end to the distal end of the tube will cause the one or more agents 162 to be expelled from the unidirectional exit port 144. Numerous types of motors 156 may be associated with one or more agent delivery devices 128. Examples of such motors 156 include, but are not limited to, stepper motors 156, osmotic motors 156, piezoelectric motors 156, ultrasonic motors 156, acoustic motors 156, and the like. In some embodiments, one or more moveable members 158 may be operably associated with one or more ratcheted members such that the one or more moveable members 158 may be engaged by the one or more ratcheted members in conjunction with movement facilitated by one or more motors 156.

### Signal

Numerous types of signals may be used in association with system 100. In some embodiments, a signal may be an internal signal 160. In some embodiments, a signal may be an external sensor signal 166. In some embodiments, a signal may be an external device signal 170. Examples of such signals include, but are not limited to, analog signals, digital signals, acoustic signals, optical signals, radio signals, wireless signals, hardwired signals, infrared signals, ultrasonic signals, and the like. In some embodiments, one or more signals may not be encrypted. In some embodiments, one or more signals may be encrypted. In some embodiments, one or more signals may be sent through use of a secure mode of transmission. In some embodiments, one or more signals may be coded for receipt by a specific individual. In some embodiments, such code may include anonymous code that is specific for an individual. Accordingly, information included within one or more signals may be protected against being accessed bad others who are not the intended recipient.

In some embodiments, one or more signals may include information associated with the operation of one or more agent delivery devices 128. In some embodiments, one or more signals may include information associated with the operation of one or more motors 156 associated with an agent delivery device 128. For example, in some embodiments, one or more signals may include information associated with the operation of one or more stepper motors 156 associated with an agent delivery device 128. Examples of such information include, but are not limited to, the number of cycles that a motor 156 is to operate, the number of steps that a motor 156 is to operate, the duration of time for which a motor 156 is to operate, the rate at which a motor 156 is to operate, one or more times when a motor 156 is to operate, and the like. Such information may be associated with numerous types of motors 156. In some embodiments, one or more signals may include information that is associated with the operation of one or more ports that are associated with one or more agent delivery devices 128. In some embodiments, one or more signals may include instructions for an agent delivery device 128 to open one or more ports. In some embodiments, one or more signals may include instructions for an agent delivery device 128 to close one or more ports. Examples of such ports include, but are not limited to, electromagnetic ports, shape memory ports, and the like (e.g., Low et al., Sensors and Actuators B: Chemical, 76:149-160 (2000), Pan et al., Proceedings of the 26^{th} Annual International Conference of the IEEE EMBS, San Francisco, Calif., USA, Sep. 1-5 (2004), U.S. Pat. No. 6,454,759). In some embodiments, such ports may be associated with one or more osmotic motors 156. In some embodiments, one or more ports may be opened and/or closed to regulate entry of fluid into one or more chambers of an osmotic motor 156 to control the operation of the motor 156. For example, in some embodiments, one or more ports may be opened to allow fluid to enter into one or more chambers of an osmotic motor 156 to facilitate movement of one or more moveable members 158 that facilitate extrusion of one or more agents 162 from the agent delivery device 128. The one or more ports may be maintained in an open position to provide for entry of fluid into one or more chambers of the osmotic motor 156 or the ports my be closed to disallow entry of fluid into one or more chambers of the osmotic motor 156. Accordingly, in some embodiments, one or more signals may be received by one or more agent delivery devices 128 that provide the one or more agent delivery devices 128 with instructions associated with the delivery of one or more agents 162.

### Electromagnetic Energy

Electrical power may be electromagnetically coupled from one or more electromagnetic transmitters 172 with one or more electromagnetic receivers (e.g., sensor electromagnetic receiver 118 and/or agent delivery electromagnetic receiver 132). Accordingly, electrical power that is transferred to the one or more electromagnetic receivers may be used to power one or more operably linked sensors 102 and/or agent delivery devices 128. Electromagnetic transmitters 172 that may be modified to transmit electrical power to a sensor 102 and/or agent delivery device 128 have been described (e.g., U.S. Pat. No. 5,571,152).

### External Interface

In some embodiments, system 100 may include one or more external interfaces 168. In some embodiments, one or more external interfaces 168 may be configured to transmit one or more external device signals 170. In some embodiments, one or more external interfaces 168 may be configured to transmit one or more external sensor signals 166. In some embodiments, one or more external interfaces 168 may be configured to receive one or more external device signals 170. In some embodiments, one or more external interfaces 168 may be configured to receive one or more external sensor signals 166. In some embodiments, one or more external interfaces 168 may be configured to transmit electromagnetic energy 164.

Numerous types of electromagnetic transmitters 172 may be associated with one or more external interfaces 168. Methods to construct electromagnetic transmitters 172 have been described (e.g., U.S. Pat. No. 5,571,152). Briefly, in some embodiments, the electromagnetic transmitter 172 may include a ferrite core around which is wrapped an electrical conductor. Other types of material having high magnetic permeability and relatively low magnetic hysteresis may be used for the core. Insulating tape may be wrapped around the electrical conductor, or the electromagnetic transmitter 172 may be dipped in a resin to form a coating that stabilizes and fixes the electrical conductor on the core. A return lead from one end of the electrical conductor may include one of two leads that are coupled to an AC power supply.

Numerous types of recording units 178 may be associated with one or more external interfaces 168. Examples of such recording units 178 include, but are not limited to, many types of memory, optical disks, magnetic disks, magnetic tape, and the like. In some embodiments, one or more recording units 178 provide for user interaction.

Numerous types of user interfaces 176 may be associated with one or more external interfaces 168. A user may interact with one or more external interfaces 168 through use of numerous technologies. For example, user interaction can occur through use of hardwired methods, such as through use of a keyboard, use of wireless methods, use of the internet, and the like.

Numerous types of display units 174 may be associated with one or more external interfaces 168. Examples of such display units 174 include, but are not limited to, passive displays, active displays, light emitting diodes, liquid crystal displays, and the like.

An external interface 168 may include one or more external transmitters 180. Numerous types of external transmitters 180 may be used in association with an external interface 168. Examples of such external transmitters 180 include, but are not limited to, transmitters that transmit one or more acoustic signals, optical signals, radio signals, wireless signals, hardwired signals, infrared signals, ultrasonic signals, and the like (e.g., U.S. Pat. Nos. RE39,785; 7,260,768; 7,260,764; 7,260,402; 7,257,327; 7,215,887; 7,218,900; herein incorporated by reference). In some embodiments, one or more external transmitters 180 may transmit one or more signals that are encrypted. Numerous types of transmitters are known and have been described (e.g., U.S. Pat. Nos. and Published U.S. Patent Applications: 7,936,595; 7,260,155; 7,297,956; US2006/0280307; herein incorporated by reference).

An external interface 168 may include one or more external receivers 182. Numerous types of external receivers 182 may be used in association an external interface 168. Examples of such external receivers 182 include, but are not limited to, receivers that receive one or more acoustic signals, optical signals, radio signals, wireless signals, hardwired signals, infrared signals, ultrasonic signals, and the like. Such receivers are known and have been described (e.g., U.S. Pat. Nos. RE39,785; 7,218,900; 7,254,160; 7,245,894; 7,206,605 herein incorporated by reference).

### Agent

Numerous types of agents 162 may be used within system 100. Examples of such agents 162 include, but are not limited to, pharmaceutical agents, hormones, cytokines, and the like. Examples of pharmaceutical agents include, but are not limited to, ace-inhibitors, alpha-adrenergic agonists, beta-adrenergic agonists, alpha-adrenergic blockers, beta-adrenergic blockers, adrenocortical steroids, adrenocortical suppressants, adrenocortical hormones, alcohol deterrents, aldose reductase inhibitors, aldosterone antagonists, AMPA receptor antagonists, anabolics, analeptics, analgesics, angrogens, anesthetics, angiotensin II receptor antagonists, anorexics, anthelmintics, antiallergics, antialopecia agents, antiamebics, antiandrogens, antianginals, antiarrhythmics, antiarteriosclerotics, antiarthritics, antirheumatics, antiasthmatics, antibacterials, antibacterial adjuvants, antibiotics, anticholelithogenics, anticholesteremics, anticholinergics, anticoagulants, anticonvrulsants, antidepressants, antidiabetics, antidiarrheals, antidiuretics, antidyskinetics, antieczematics, antiemetics, antiestrogens, antifibrotics, antifungals, antiglaucoma agents, antigonadotropins, antiagout agents, antihemophilic factors, antihemorrhagics, antihistaminics, antihypercholesterolemics, antihyperlipidemics, antihyperparathyroids, antihyperphosphatemics, antihypertensives, antihyperthroids, antihypotensives, antihypothyroids, anti-inflammatory agents, antimalarials, antimanics, antimethemoglobinemics, antimigraines, antimuscarinics, antimycotics, antinauseants, antineoplastics, antineoplastic adjuvants, antineurtropenics, antiobesity agents, antiobsessionals, antiosteoporotics, antipagentics, antiparkinsonian agents, antiperistaltics, antipheochromocytomas, antipheumocystics, antiprogestins, antiprostatic hypertrophy agents, antiprotozoals, antipuritics, antipsoriatics, antipsychotics, antipyretics, antirickettsials, antiseborrheics, antisepsis agents, antispasmodics, antisyphilitics, antithrombotics, antithrombocythemics, antitubercular agents, antitussives, antiulceratives, antiurolithics, antivenins, antivirals, anxiolytic agents, aromatase inhibitors, atriopeptidase inhibitors, benzodiazepine antagonists, beta-blockers, bone resorption inhibitors, bradycardic agents, bradykinin antagonists, bronchodilators, calcium channel blockers, calcium regulators, carbonic anhydrase inhibitors, cardiac depressants, cardioprotective agents, cardiotonics, CCK antagonists, cholelitholytic agents, choleretics, cholinergics, cholinesterase inhibitors, cholinesterase reactivators; central nervous system stimulants, COMT inhibitors, contraceptives, cyclooxygenase-2 inhibitors, cytoprotectants, debriding agents, decongestants, dental plague inhibitors, depigmentors, dermatitis herpetiformis suppressants, diuretics, dopamine receptor agonists, endothelial receptor antagonists, enkephalinase inhibitors, estrogens, estrogen antagonists, fibrinogen receptor antagonists, gastric and pancreatic secretion stimulants, gastric proton pump inhibitors, gastric secretion inhibitors, gastroprokinetics, glucocorticoids, alpha-glucosidase inhibitors, gonad-stimulating principles, growth hormone antagonists, growth hormone inhibitors, growth hormone releasing factors, growth stimulants, hematinics, hematopoietics, hemostatics, hepatoprotectants, histamine H1-receptor antagonists, human immunodeficiency virus fusion inhibitors, human immunodeficiency virus protease inhibitors, immunomodulators, immunosuppressants, insulin sensitizers, lactation stimulating hormones, leukotriene antagonists, LH-RH agonists, LH-RH antagonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuraminidase inhibitors, neuromuscular blocking agents, neutral endopeptidase inhibitors, neuroprotective agents, NMDA receptor antagonists, nootropic, ovarian hormones, oxytocic agents, pepsin inhibitors, phosphodiesterase inhibitors, platelet activating factor antagonists, potassium channel activators, potassium channel blockers, progestogens, prolactin inhibitors, prostaglandins, prostaglandin analogs, protease inhibitors, proton pump inhibitors, pulmonary surfactants, 5-alpha-reductase inhibitors, respiratory stimulants, reverse transcriptase inhibitors, scabicides, sedatives, hypnotics, serotonin noradrenaline reuptake inhibitors, serotonin receptor agonists, serotonin receptor antagonists, serotonin reuptake inhibitors, sialagogues, somatostatin analogs, thromboxane A2-receptor antagonists, thromboxane A2-synthetase inhibitors, thyroid hormones, thyroid inhibitors, thyrotropic hormones, tocolytics, topoisomerase inhibitors, vasodilators, vasopeptidase inhibitors, vasoprotectants, vitamins, vulnerary agents, Wilson's disease treatments, xanthine oxidase inhibitors, nitric oxide, nitric oxide donors, or substantially any combination thereof.

Examples of hormones include, but are not limited to, estrogen, glucagon-like peptides, growth hormone, melatonin, serotonin, thyroxine, triiodothyronine, epinephrine, norepinephrine, dopamine, antimullerian hormone, adiponectin, adrenocorticotropic hormone, angiotensin, vasopressin, atriopeptin, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, somatomedin, leptin, luteinizing hormone, melanocyte stimulating hormone, oxytocin, parathyroid hormone, prolactin, relaxin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estradiol, estrone, estriol, progesterone, calcitrol, calcidiol, prostaglandins, leukotrienes, prostacyclin, thromboxane, prolactin releasing hormone, lipotropin, brain natriuretic peptide, neuropeptide Y, histamine, endothelin, renin, enkephalin, or substantially any combination thereof.

Examples of cytokines include, but are not limited to, bone morphogenic proteins, brain-derived neurotrophic factor, interleukin 2, interleukin 3, interleukin 6, interleukin 7, interleukin 10, interleukin 11, interleukin 12, interleukin 18, angiostatin, Apo2L, ciliary neurotrophic factor, cardiotrophin-1, epidermal growth factor, erythropoietin, insulin-like growth factors, interferon, leptin, macrophage stimulating protein, nerve growth factor, neurotrophin 3, neurotrophin 4, oncostatin M, or substantially any combination thereof.

Following are a series of flowcharts depicting implementations. For ease of understanding, the flowcharts are organized such that the initial flowcharts present implementations via an example implementation and thereafter the following flowcharts present alternate implementations and/or expansions of the initial flowchart(s) as either sub-component operations or additional component operations building on one or more earlier-presented flowcharts. Those having skill in the art will appreciate that the style of presentation utilized herein (e.g., beginning with a presentation of a flowchart(s) presenting an example implementation and thereafter providing additions to and/or further details in subsequent flowcharts) generally allows for a rapid and easy understanding of the various process implementations. In addition, those skilled in the art will further appreciate that the style of presentation used herein also lends itself well to modular and/or object-oriented program design paradigms.

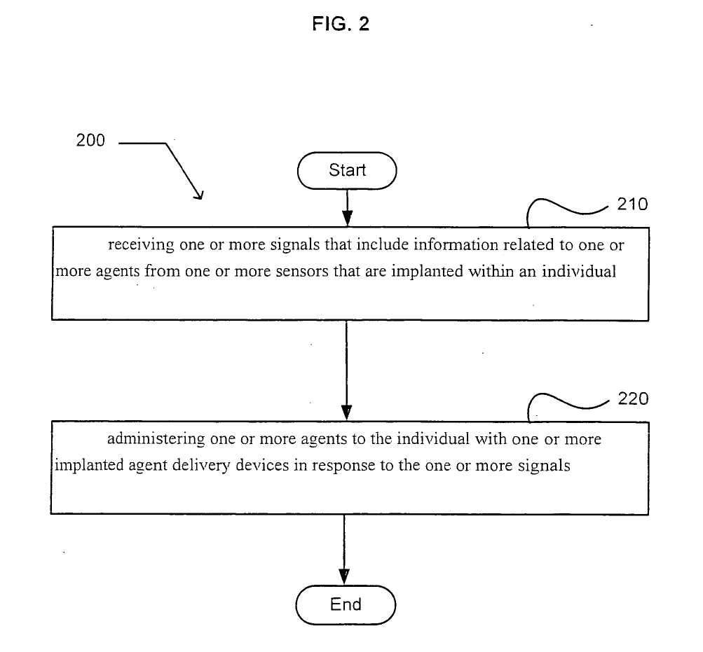

FIG. 2 illustrates an operational flow 200 representing examples of operations that are related to the performance of a method for receiving one or more signals that include information related to one or more agents from one or more sensors that are implanted within an individual and administering one or more agents to the individual with one or more implanted agent delivery devices in response to the one or more signals. In FIG. 2 and in following figures that include various examples of operations used during performance of the method, discussion and explanation may be provided with respect to any one or combination of the above-described examples of FIG. 1, and/or with respect to other examples and contexts. However, it should be understood that the operations may be executed in a number of other environments and contexts, and/or modified versions of FIG. 1. Also, although the various operations are presented in the sequence(s) illustrated, it should be understood that the various operations may be performed in other orders than those which are illustrated, or may be performed concurrently.

After a start operation, the operational flow 200 includes a receiving operation 210 involving receiving one or more signals that include information related to one or more agents from one or more sensors that are implanted within an individual. In some embodiments, one or more implanted agent delivery devices 128 may receive one or more signals that include information related to one or more agents 162 from one or more sensors 102 that are implanted within an individual. Numerous types of signals may be received by one or more agent delivery devices 128. Examples of such signals include, but are not limited to, acoustic signals, infrared signals, ultrasonic signals, optical signals, radio signals, and the like. In some embodiments, one or more agent delivery devices 128 may receive one or more signals directly from one or more implanted sensors 102. For example, in some embodiments, one or more receivers associated with one or more implanted agent delivery devices 128 may receive one or more signals that are transmitted from one or more transmitters that are associated with one or more implanted sensors 102. In some embodiments, one or more agent delivery devices 128 may receive one or more signals indirectly from one or more implanted sensors 102. For example, in some embodiments, one or more receivers associated with one or more implanted agent delivery devices 128 may receive one or more signals that are transmitted by one or more implanted devices that receive one or more signals from one or more implanted sensors 102. In some embodiments, one or more agent delivery devices 128 may receive one or more signals that include numerous types of information. Examples of such information include, but are not limited to, one or more concentrations of one or more agents 162, one or more changes in the concentration of one or more agents 162, one or more changes in the concentration of one or more agents 162 relative to time, and the like. In some embodiments, one or more agent delivery devices 128 may receive one or more signals that include instructions for administration of one or more agents 162. Such instructions may include, but are not limited to, one or more amounts of one or more agents 162 to be administered, one or more times when one or more agents 162 are to be delivered, one or more durations for administration of one or more agents 162, and the like. In some embodiments, one or more agent delivery devices 118 may receive one or more signals that include instructions associated with operation of one or more motors 156 associated with the one or more agent delivery devices 128. For example, in some embodiments, one or more signals may instruct one or more stepper motors 156 associated with an agent deliver), device 128 to operate for a selected number of cycles. Accordingly, in some embodiments, the amount of one or more agents 162 that are administered by an agent delivery device 128 may be correlated to the number of cycles that a stepper motor 156 is operated to drive a moveable member 158 and cause extrusion of the one or more agents 162 from the agent delivery device 128. Accordingly, such correlations may be made with numerous types of motors 156 (e.g., piezoelectric motors 156, stepper motors 156, squiggle motors 156, and the like).

After a start operation, the operational flow 200 includes an administering operation 220 involving administering one or more agents to the individual with one or more implanted agent delivery devices in response to the one or more signals. In some embodiments, one or more agents 162 may be delivered to the individual with one or more implanted agent delivery devices 128 in response to the one or more signals. In some embodiments, one or more agent delivery devices 128 may administer one or more selected agents 162 in response to one or more signals. For example, in some embodiments, an agent delivery device 128 may be configured to administer more than one type of agent 162. Accordingly, in some embodiments, an agent delivery device 128 may receive one or more signals that instruct the agent delivery device 128 to administer one or more selected agents 162.

In some embodiments, an agent delivery device 128 may act in association with an implanted sensor 109 to calibrate the sensor 102 to administer one or more agents 162 to an individual. For example, in some embodiments, a sensor 102 may detect the concentration of an agent 162 in the blood stream of an individual. The sensor 102 may then transmit one or more signals that instruct a stepper motor 156 associated with an agent delivery device 128 to turn through three rotations to cause movement of a moveable member 158 which results in administration of an agent 162 to the individual from the agent delivery device 128. The implanted sensor 102 may then determine the concentration of the administered agent 162 within the bloodstream of the individual following administration of the agent 162. In such a manner, a sensor 102 may correlate the number of rotations of a stepper motor 156 associated with an agent delivery device 128 to the concentration of agent 162 that is administered to an individual. Accordingly, the calibrated sensor 102 may then transmit one or more signals that instruct an agent delivery device 198 to administer a predetermined amount of agent 162 to an individual. In some embodiments, one or more calibrated sensors 102 may be used to maintain the concentration of one or more agents 162 substantially at a selected concentration setpoint within an individual (e.g., plasma concentration of an agent 162). In some embodiments, one or more calibrated sensors 102 may be used to maintain the concentration of one or more agents 162 substantially within a selected concentration range within an individual (e.g., plasma concentration of an agent 162). In some embodiments, one or more sensors 102 may act in association with one or more, agent delivery devices 128 during the calibration process.

In some embodiments, an implanted agent delivery device 128 may act in association with an implanted sensor 102 to calibrate the agent delivery device 128 to administer one or more agents 162 to an individual. For example, in some embodiments, a sensor 102 may detect the concentration of an agent 162 in the bloodstream of an individual. The sensor 102 may then transmit one or more signals that are received by an agent delivery device 128. The agent delivery device 128 may then cause a stepper motor 156 associated with the agent delivery device 128 to turn through three rotations to cause movement of a moveable member 158 which results in administration of an agent 162 to the individual. The implanted sensor 102 may then transmit one or more signals that include information associated with the concentration of the administered agent 162 within the bloodstream of the individual following administration of the agent 162. The one or more signals may be received by the agent delivery device 128 which may then correlate the number of rotations of the stepper motor 156 associated with the agent delivery device 128 to the concentration of agent 162 that is administered to an individual. In some embodiments, one or more calibrated agent delivery devices 128 may be used to maintain the concentration of one or more agents 162 substantially at a selected concentration setpoint within an individual (e.g., plasma concentration of an agent 162). In some embodiments, one or more calibrated agent delivery devices 128 may be used to maintain the concentration of one or more agents 162 substantially within a selected concentration range within an individual (e.g., plasma concentration of an agent 162). In some embodiments, one or more sensors 102 may act in association with one or more agent delivery devices 128 during the calibration process.

In some embodiments, one or more sensors 102 and one or more agent delivery devices 128 may act in association to maintain one or more agent 162 concentrations substantially at one or more setpoints within an individual. For example, in some embodiments, one or more sensors 102 may transmit one or more signals at time intervals that include information associated with the concentration of one or more agents 162 within an individual. In some embodiments, such signals may be received by one or more agent delivery devices 128 that may administer the one or more agents 162 in response to the one or more signals. For example, in some embodiments, if one or more sensors 102 detect that one or more agents 162 are within a selected concentration range, then one or more agent delivery devices 128 that receive the one or more signals will not administer the one or more agents 162 to the individual. However, in some embodiments, if one or more sensors 102 detect that one or more agents 162 are below a selected concentration range, then one or more agent delivery devices 128 that receive the one or more signals will administer the one or more agents 162 to the individual.

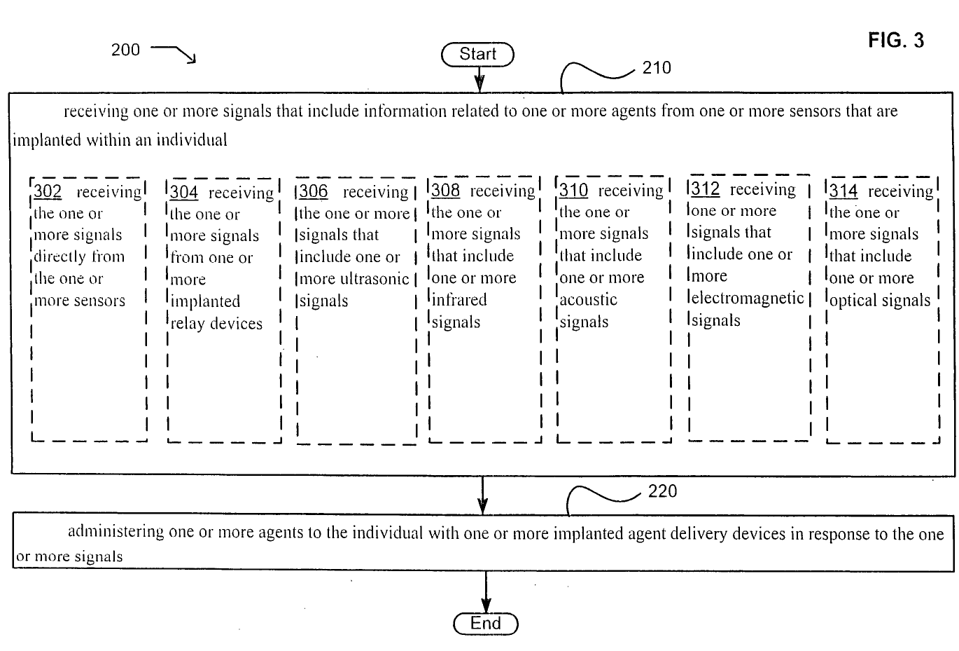

FIG. 3 illustrates alternative embodiments of the example operational flow 200 of FIG. 2. FIG. 3 illustrates example embodiments where the receiving operation 210 may include at least one additional operation. Additional operations may include an operation 302, operation 304, operation 306, operation 308, operation 310, operation 312, and/or operation 314.

At operation 302, the receiving operation 210 may include receiving the one or more signals directly from the one or more sensors. In some embodiments, one or more implanted agent delivery devices 128 may receive the one or more signals directly from the one or more sensors 102. Numerous types of signals may be received. In some embodiments, one or more receivers associated with one or more agent delivery devices 128 that are implanted within an individual may receive one or more signals that are transmitted by one or more transmitters that are associated with one or more sensors 102 that are implanted within the individual. For example, in some embodiments, one or more implanted agent delivery devices 128 may receive one or more acoustic signals that are transmitted by one or more acoustic transmitters associated with one or more implanted sensors 102. Methods to fabricate acoustic transmitters and acoustic receivers have been described (e.g., MicroChips Inc., Bedford, Mass., U.S. Pat. No. 7,198,603). In some embodiments, one or more signals may include information associated with the operation of one or more agent delivery devices 128. In some embodiments, one or more signals may include information associated with the operation of one or more motors 156 associated with an agent delivery device 128. For example, in some embodiments, one or more signals may include information associated with the operation of one or more stepper motors 156 associated with an agent delivery device 128. Examples of such information include, but are not limited to, the number of cycles that a motor 156 is to operate, the number of steps that a motor 156 is to operate, the duration of time for which a motor 156 is to operate, the rate at which a motor 156 is to operate, one or more times when a motor 156 is to operate, and the like. Such information may be associated with numerous types of motors 156. In some embodiments, one or more signals may include information that is associated with the operation of one or more points that are associated with one or more agent delivery devices 128. In some embodiments, one or more signals may include instructions for an agent delivery device 128 to open one or more ports. In some embodiments, one or more signals may include instructions for an agent delivery device 128 to close one or more ports. Examples of such ports include, but are not limited to, electromagnetic ports, shape memory ports, and the like (e.g., Low et al., Sensors and Actuators B: Chemical, 76:149-160 (9000), Pan et al., Proceedings of the 26^{th} Annual International Conference of the IEEE EMBS, San Francisco, Calif., USA, Sep. 1-5 (2004), U.S. Pat. No. 6,454,759). In some embodiments, such ports may be associated with one or more osmotic motors 156. In some embodiments, one or more ports may be opened and/or closed to regulate entry of fluid into one or more chambers of an osmotic motor 156 to control the operation of the motor 156. For example, in some embodiments, one or more ports may be opened to allow fluid to enter into one or more chambers of an osmotic motor 156 to facilitate movement of one or more moveable members 158 that facilitate extrusion of one or more agents 162 from the agent delivery device 128. The one or more ports may be maintained in an open position to provide for entry of fluid into one or more chambers of the osmotic motor 156 or the ports may be closed to disallow entry of fluid into one or more chambers of the osmotic motor 156. Accordingly, in some embodiments, one or more signals may be received by one or more agent delivery devices 128 that provide the one or more agent delivery devices 128 with instructions associated with the delivery of one or more agents 162.

At operation 304, the receiving operation 210 may include receiving the one or more signals from one or more implanted relay devices. In some embodiments, one or more implanted agent delivery devices 128 may receive the one or more signals from one or more implanted relay devices. In some embodiments, a sensor 102 may transmit one or more signals that are received by one or more relay devices that transmit one or more signals that are received by one or more agent delivery devices 128. Numerous types of signals may be transmitted. In some embodiments, a relay device may receive one or more types of signals and transmit one or more other types of signals. For example, in some embodiments, one or more relay devices may receive one or more optical signals and then transmit one or more acoustic signals.

At operation 306, the receiving operation 210 may include receiving the one or more signals that include one or more ultrasonic signals. In some embodiments, one or more implanted agent delivery devices 128 may receive the one or more signals that include one or more ultrasonic signals. Numerous types of ultrasonic transmitters and receivers may be used to send and receive signals. Methods to fabricate ultrasonic transmitters and receivers are known and have been described (e.g., U.S. Pat. Nos. 7,162,930; 6,854,338; 6,087,760; 6,212,936; 4,326,274; and 5,483,226).

At operation 308, the receiving operation 210 may include receiving the one or more signals that include one or more infrared signals. In some embodiments, one or more implanted agent delivery devices 128 may receive the one or more signals that include one or more infrared signals. Numerous types of infrared transmitters and receivers may be used to send and receive signals. Methods to fabricate infrared transmitters and receivers are known and have been described (e.g., U.S. Pat. Nos. 4,371,814; 5,359,448; and 5,331,450).

At operation 310, the receiving operation 210 may include receiving the one or more signals that include one or more acoustic signals. In some embodiments, one or more implanted agent delivery devices 128 may receive the one or more signals that include one or more acoustic signals. Numerous types of acoustic transmitters and receivers may be used to send and receive signals. Methods to fabricate infrared transmitters and receivers are known and have been described (e.g., U.S. Pat. Nos. 7,301,473; 4,149,478; 3,978,940; 4,002,897; and 6,488,116).

At operation 312, the receiving operation 210 may include receipting the one or more signals that include one or more electromagnetic signals. In some embodiments, one or more implanted agent delivery devices 128 may receive the one or more signals that include one or more electromagnetic signals. In some embodiments, one or more electromagnetic signals may be received through use of an optical fiber (e.g., U.S. Pat. No. 5,307,195). In some embodiments, one or more electromagnetic signals may be received through use of a conductive wire (e.g., U.S. Pat. No. 5,122,773). Devices that are configured to receive one or more electromagnetic signals have been described (e.g., U.S. Pat. No. 6,993,259).

At operation 314, the receiving operation 210 may include receiving the one or more signals that include one or more optical signals. In some embodiments, one or more implanted agent delivered devices 128 may receive the one or more signals that include one or more optical signals. Numerous types of optical transmitters and receivers malt be used to send and receive signals. Methods to fabricate optical transmitters and receivers are known and have been described (e.g., U.S. Pat. Nos. 5,170,274; 5,949,566; 6,192,060; 5,307,196; and 6,304,357).

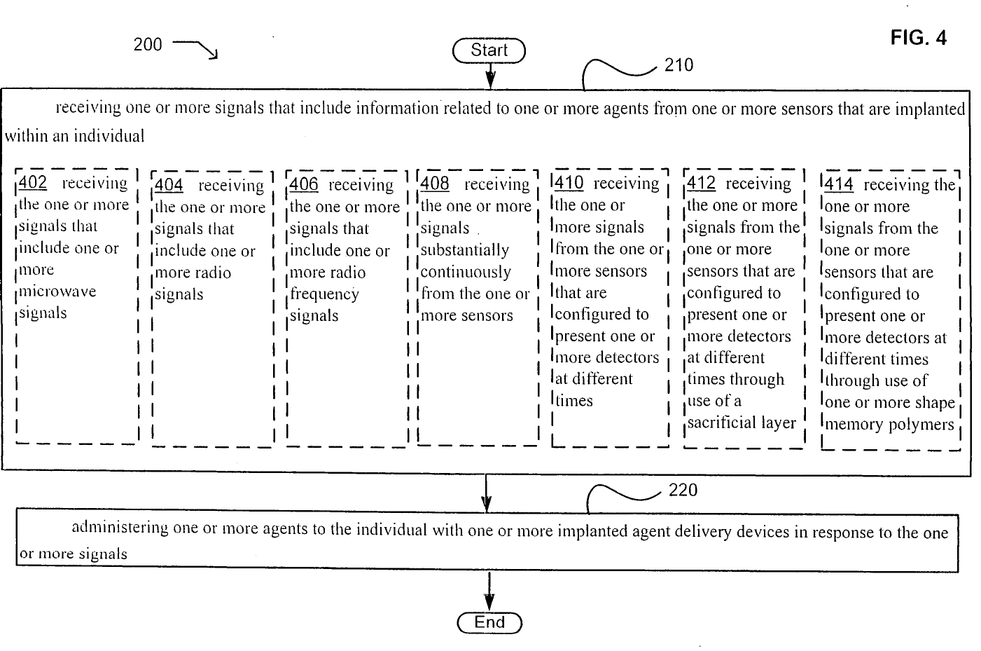

FIG. 4 illustrates alternative embodiments of the example operational flow 200 of FIG. 2. FIG. 4 illustrates example embodiments where the receiving operation 210 may include at least one additional operation. Additional operations may include an operation 402, operation 404, operation 406, operation 408, operation 410, operation 412, and/or operation 414.

At operation 402, the receiving operation 210 may include receiving the one or more signals that include one or more microwave signals. In some embodiments, one or more implanted agent delivery devices 128 may receive the one or more signals that include one or more microwave signals. Devices that are configured to transmit and receive one or more microwave signals have been described (e.g., U.S. Pat. Nos. 4,196,393; 4,032,859; 4,121,163; 5,053,792).

At operation 404, the receiving operation 210 may include receiving the one or more signals that include one or more radio signals. In some embodiments, one or more implanted agent deliver), devices 128 mats receive the one or more signals that include one or more radio signals. Numerous types of radio transmitters and receivers may be used to send and receive signals. Methods to fabricate radio transmitters and receivers are known and have been described (e.g., U.S. Pat. Nos. 5,826,177; 4,355,401; 5,941,561; and 5,353,311).

At operation 406, the receiving operation 210 may, include receiving the one or more signals that include one or more radio frequency signals. In some embodiments, one or more implanted agent delivery devices 128 may, receive the one or more signals that include one or more radio frequency signals. Methods to fabricate devices that transmit and receive radio frequency signals are known and have been described (e.g., U.S. Pat. Nos. 7,171,175; 7,031,676; 6,587,511; 4,258,436; 4,047,121; 4,013,966).

At operation 408, the receiving, operation 210 may include receiving the one or more signals substantially continuously from the one or more sensors. In some embodiments, one or more implanted agent delivered devices 128 may receive the one or more signals substantially continuously from the one or more sensors 102. In some embodiments, one or more agent delivery devices 128 may receive one or more signals from one or more sensors 102 substantially continuously for a period of time.

In some embodiments, one or more agent delivery devices 128 may substantially continuously receive one or more signals during a period of time when the agent delivery device 128 is administering one or more agents 162. For example, in some embodiments, the concentration of an agent 162 within an individual may be less than a setpoint and/or concentration range where the agent 162 is to be substantially maintained. Accordingly, one or more sensors 102 may detect that the agent 162 is at a low concentration and substantially continuously transmit one or more signals associated with the concentration of the agent 162 that are received by, one or more agent delivery devices 128. The agent delivery device 128 may then administer the agent 1662 to the individual until the sensor 102 determines that the concentration of the agent 162 within the individual has substantially reached the setpoint. The sensor 102 may then stop substantially continuously transmitting one or more signals until the concentration of the agent 162 again decreases below a setpoint and/or concentration range when the sensor 102 will again substantially continuously transmit one or more signals. In such embodiments, direct feedback between one or more sensors 102 and one or more agent delivery devices 128 may occur that facilitates administration of one or more agents 162 to an individual such that the concentration of the one or more agents 162 within the individual (e.g., within the bloodstream) may be maintained.

At operation 410, the receiving operation 210 may include receiving the one or more signals from the one or more sensors that are configured to present one or more detectors at different times. In some embodiments, one or more implanted agent delivery devices 128 may receive the one or more signals from the one or more sensors 102 that are configured to present one or more detectors at different times. In some embodiments, one or more sensors 102 may be configured to present one or more detectors. The detectors may be configured to detect numerous analytes. Examples of such analytes include, but are not limited to, pharmaceutical agents 162, hormones, cytokines, chemokines, metabolites of pharmaceutical agents 1662, products resulting from administration of one or more pharmaceutical agents 162, and the like. In some embodiments, one or more sensors 102 may be configured to include detectors that are sequestered. For example, in some embodiments, a sensor 102 may be configured to include selectively accessible sections that are configured to enclose one or more detectors. In such embodiments, the selectively accessible sections may be sealed such that the detectors contained within the selectively accessible sections are sequestered from the outside environment. In some embodiments, the selectively accessible sections may be unmasked to expose one or more detectors enclosed therein to the outside environment. Accordingly, one or more detectors may be unmasked in a regulated fashion for the detection of one or more analytes at selected times. Numerous materials may be used to mask the selectively accessible sections. Examples of such materials include, but are not limited to, metal (e.g., gold foil), shape memory polymers (e.g., U.S. Pat. No. 6,454,759), and the like. Numerous types of detectors 114 may be enclosed within one or more selectively accessible sections. Examples of such detectors 114 include, but are not limited to, electrodes, surface plasmon resonance detectors 114, microelectromechanical systems detectors 114, microcantilever detectors 114, nitric oxide detectors 114, osmotic detectors 114, relativity-based detectors 114, chemical detectors 114, pressure detectors 114, electrochemical detectors 114, piezoelectric detectors 114, pH detectors 114, hydrogel detectors 114, enzymatic detectors 114, ball integrated circuit detectors 114, affinity viscosimetric detectors 114, blood pressure detectors 114; metal detectors 114, and the like (e.g., U.S. Pat. Nos. 7,162,289; 6,280,604; 5,603,820; 5,582,170; 6,287,452; 7,291,503; 6,764,446; 7,168,294; 6,823,717; 7,205,701; 6,268,161; 4,703,756; 6,965,791; 6,546,268; 6,210,396; 6,514,689; 6,234,973; 6,442,413; Tu et al., Electroanalysis, 11:70-74 (1999), Malinski et al., Molecular Mechanisms of Metal Toxicity and Carcinogenicity, Environmental Health Perspectives 102, Supplement 3, September 1994).

At operation 412, the receiving operation 210 may include receiving the one or more signals from the one or more sensors that are configured to present one or more detectors at different times through use of a sacrificial layer. In some embodiments, one or more implanted agent delivery devices 128 may receive the one or more signals from the one or more sensors 102 that are configured to present one or more detectors at different times through use of a sacrificial layer. For example, in some embodiments, one or more selectively accessible portions 112 may be covered with a gold sacrificial layer that may be removed though electrochemical dissolution with a constant DC current (e.g., 35 mL/cm²) (Pan et al., Proceedings of the 26^{th} Annual International Conference of the IEEE EMBS, San Francisco, Calif., USA, Sep. 1-5, 2004).

At operation 414, the receiving operation 10 may include receiving the one or more signals from the one or more sensors that are configured to present one or more detectors at different times through use of one or more shape memory polymers. In some embodiments, one or more implanted agent delivery devices 128 may receive the one or more signals from the one or more sensors 102 that are configured to present one or more detectors at different times through use of one or more shape memory polymers. In some embodiments, one or more selectively accessible portions 112 may be covered with a shape-memory polymer that may be activated to unsequester the one or more selectively accessible portions 112 (e.g., U.S. Pat. No. 6,454,759).

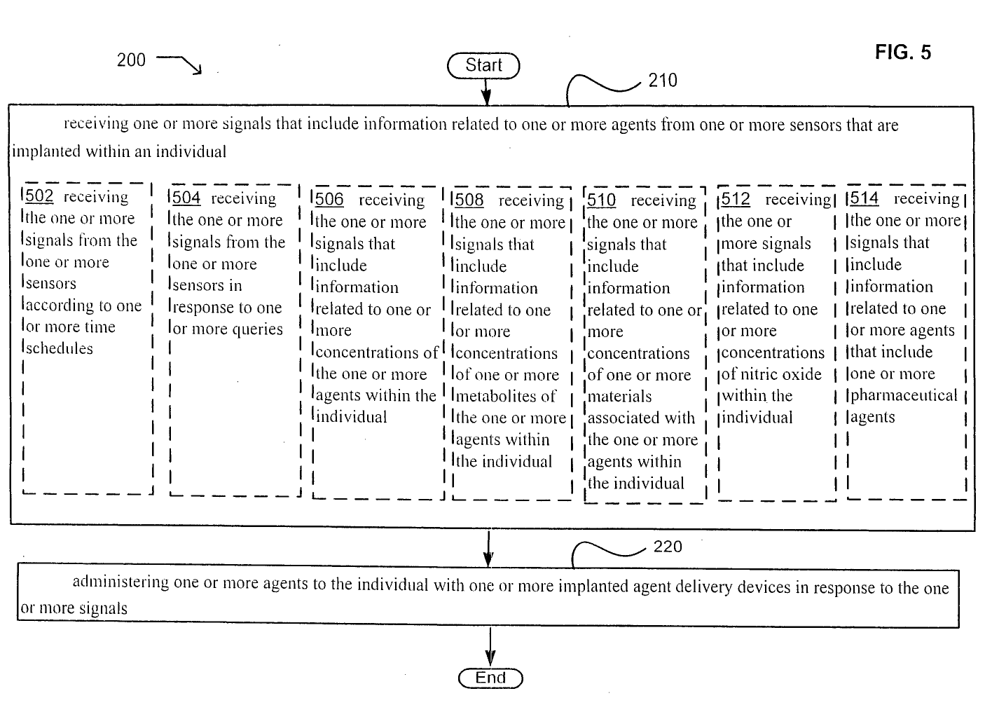

FIG. 5 illustrates alternative embodiments of the example operational flow 900 of FIG. 2. FIG. 5 illustrates example embodiments where the receiving operation 210 may include at least one additional operation. Additional operations may include an operation 502, operation 504, operation 506, operation 508, operation 510, operation 512, and/or operation 514.

At operation 502, the receiving operation 210 may include receiving the one or more signals from the one or more sensors according to one or more time schedules. In some embodiments, one or more implanted agent delivery devices 128 may receive the one or more signals from the one or more sensors 102 according to one or more time schedules. In some embodiments, one or more agent delivery devices 128 may receive one or more signals from one or more sensors 102 that are programmed to transmit one or more signals according to one or more time schedules. In some embodiments, one or more sensors 102 may be programmed to detect one or more agents 162 and then transmit one or more signals according to a time schedule. In some embodiments, one or more agent delivery devices 128 may be programmed to receive one or more signals from one or more sensors 102 according to a time schedule.

At operation 504, the receiving operation 210 malt include receiving the one or more signals from the one or more sensors in response to one or more queries. In some embodiments, one or more implanted agent delivery devices 128 may receive the one or more signals from the one or more sensors 109 in response to one or more queries. In some embodiments, one or more implanted agent delivery devices 128 may be programmed to query one or more sensors 102. For example, in some embodiments, one or more agent delivery devices 128 may be programmed to query one or more sensors 102 according to a time schedule. Accordingly, in some embodiments, one or more sensors 102 may be configured to detect one or more agents 162 in response to a query received from one or more agent delivery devices 128 and then transmit one or more signals that are received by one or more agent delivery devices 128. In some embodiments, one or more sensors 102 may be configured to detect one or more agents 162 in response to a query received from one or more external devices and then transmit one or more signals that are received by one or more agent delivery devices 128.

At operation 506, the receiving operation 210 may include receiving the one or more signals that include information related to one or more concentrations of the one or more agents within the individual. In some embodiments, one or more implanted agent delivery devices 128 may receive the one or more signals that include information related to one or more concentrations of the one or more agents 162 within the individual. In some embodiments, one or more implanted agent delivery devices 128 may receive one or more signals that include information related to the concentration of one or more analytes at a single time. In some embodiments, one or more implanted agent delivery devices 128 may receive one or more signals that include information related to the concentration of one or more analytes at one or more times. In some embodiments, one or more implanted agent delivery devices 128 may receive one or more signals that include information related to one or more changes in the concentration of one or more analytes at one or more times.

At operation 508, the receiving operation 210 may include receiving the one or more signals that include information related to one or more concentrations of one or more metabolites of the one or more agents within the individual. In some embodiments, one or more implanted agent delivery devices 128 may receive the one or more signals that include information related to one or more concentrations of one or more metabolites of the one or more agents 162 within the individual. In some embodiments, one or more implanted agent delivery devices 128 may receive one or more signals that include information related to one or more concentrations of one or more metabolites of the one or more agents 162 within an individual at a single time. In some embodiments, one or more implanted agent delivery devices 128 may receive one or more signals that include information related to one or more concentrations of one or more metabolites of the one or more agents 162 within an individual at one or more times. In some embodiments, one or more implanted agent delivery devices 128 may receive one or more signals that include information related to changes in the concentration of one or more metabolites of the one or more agents 162 within an individual.

At operation 510, the receiving operation 210 may include receiving the one or more signals that include information related to one or more concentrations of one or more materials associated with the one or more agents within the individual. In some embodiments, one or more implanted agent delivery devices 128 may receive the one or more signals that include information related to one or more concentrations of one or more materials associated with the one or more agents 169 within the individual. In some embodiments, one or more implanted agent delivery devices 128 may receive one or more signals that include information related to one or more concentrations of one or more materials associated with the one or more agents 162 within an individual at a single time. In some embodiments, one or more implanted agent delivery devices 128 may receive one or more signals that include information related to one or more concentrations of one or more materials associated with the one or more agents 162 within an individual at one or more times. In some embodiments, one or more implanted agent delivery devices 128 may receive one or more signals that include information related to changes in the concentration of one or more materials associated with the one or more agents 162 within an individual. Numerous materials associated with one or more agents 162 may be detected within an individual. For example, in some embodiments, an agent 162 may be an enzyme inhibitor. Accordingly, in such embodiments, materials associated with the agent 162 may include substrates utilized by the enzyme. In such embodiments, materials associated with the agent 162 may include products that are produced bad the enzyme. Examples of materials include but are not limited to substrates, products, gases nucleic acids proteins, cytokines, hormones, and the like.

At operation 512 the receiving operation 210 mail include receiving the one or more signals that include information related to one or more concentrations of nitric oxide within the individual. In some embodiments one or more implanted agent delivery devices 128 may receive the one or more signals that include information related to one or more concentrations of nitric oxide within the individual. In some embodiments, one or more implanted agent delivery devices 128 may receive one or more signals that include information related to one or more concentrations of nitric oxide within an individual at a single time. In some embodiments, one or more implanted agent delivery devices 128 may receive one or more signals that include information related to one or more concentrations of nitric oxide within an individual at one or more times. In some embodiments, one or more implanted agent delivery devices 128 may receive one or more signals that include information related to changes in the concentration of nitric oxide within the individual. In some embodiments, one or more implanted agent delivery devices 128 may receive one or more signals from one or more nitric oxide sensors 102 that are implanted within the genital region of an individual. For example, in some embodiments, one or more nitric oxide sensors 102 may be configured for implantation within the corpus cavernosum. In some embodiments, such implants may be configured as a stent (e.g., U.S. Pat. Nos. 7,236,891 and 6,442,413). Accordingly, in some embodiments, one or more sensors 102 and one or more agent delivery devices 128 may be configured to detect nitric oxide in the genital region of an individual and to deliver one or more nitric oxide donors and/or nitric oxide to the genital region of the individual.

At operation 514, the receiving operation 210 may include receiving the one or more signals that include information related to one or more agents that include one or more pharmaceutical agents. In some embodiments, one or more implanted agent delivery devices 128 may receive the one or more signals that include information related to one or more agents 162 that include one or more pharmaceutical agents 162. In some embodiments, one or more implanted agent delivery devices 128 may receive one or more signals that include information related to one or more concentrations of one or more pharmaceutical agents 162 within an individual. In some embodiments, one or more implanted agent delivery devices 128 mall receive one or more signals that include information related to one or more concentrations of one or more pharmaceutical agents 162 within an individual at a single time. In some embodiments, one or more implanted agent delivery devices 128 may receive one or more signals that include information related to one or more concentrations of one or more pharmaceutical agents 162 within an individual at one or more times. In some embodiments, one or more implanted agent delivery devices 128 may receive one or more signals that include information related to one or more changes in the concentration of one or more pharmaceutical agents 162 within an individual. In some embodiments, one or more implanted agent delivery devices 128 may receive one or more signals that include information related to one or more metabolites of one or more pharmaceutical agents 162 within an individual.

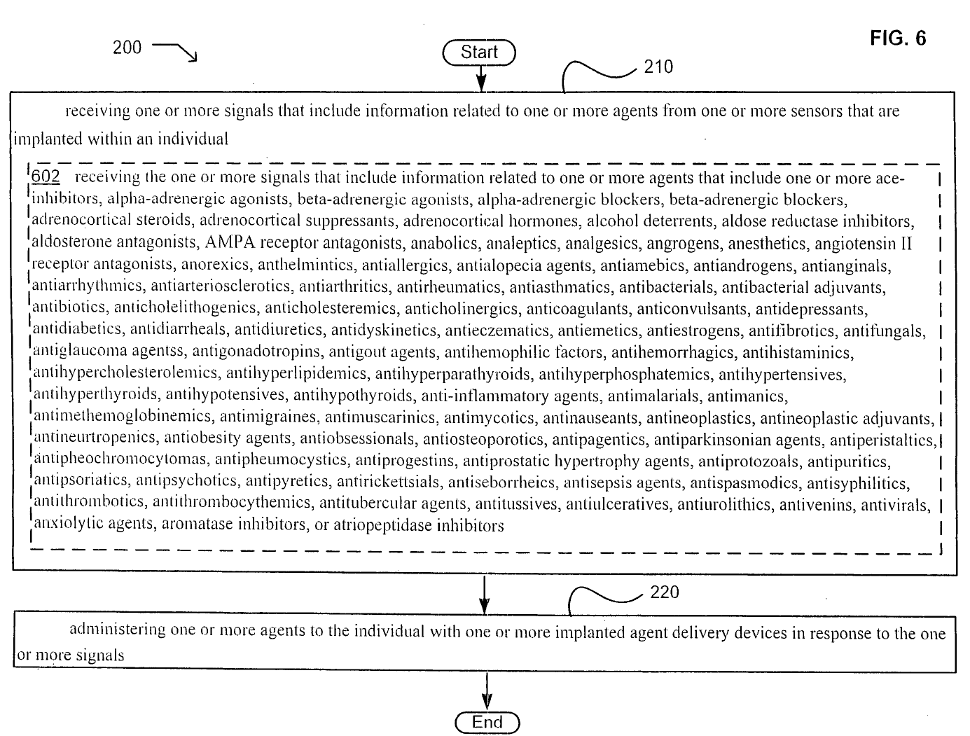

FIG. 6 illustrates alternative embodiments of the example operational flow 200 of FIG. 2. FIG. 6 illustrates example embodiments where the receiving operation 210 may include at least one additional operation. Additional operations may include an operation 602.

At operation 602, the receiving operation 210 may include receiving the one or more signals that include information related to one or more agents that include one or more ace-inhibitors, alpha-adrenergic agonists, beta-adrenergic agonists, alpha-adrenergic blockers, beta-adrenergic blockers, adrenocortical steroids, adrenocortical suppressants, adrenocortical hormones, alcohol deterrents, aldose reductase inhibitors, aldosterone antagonists, AMPA receptor antagonists, anabolics, analeptics, analgesics, angrogens, anesthetics, angiotensin II receptor antagonists, anorexics, anthelmintics, antiallergics, antialopecia agents, antiamebics, antiandrogens, antianginals, antiarrhythmics, antitarteriosclerotics, antiarthritics, antirheumatics, antiasthmatics, antibacterials, antibacterial adjuvants, antibiotics, anticholelithogenics, anti cholesteremics, anticcholinergics, anticoagulants, anticonvulsants, antidepressants, antidiabetics, antidiarrheals, antidiuretics, antidyskinetics, antieczematics, antiemetics, antiestrogens, antifibrotics, antifungals, antiglaucoma agents, antigonadotropins, antigout agents, antihemophilic factors, antihemorrhagics, antihistaminics, antihypercholesterolemics, antihyperlipidemics, antihyperparathyroids, antihyperphosphatemics, antihypertensiles, antihyperthyroids, antihypotensives, antihypothyroids, anti-inflammatory agents, antimalarials, antimanics, antimethemoglobinemics, antimigraines, antimuscarinics, antimycotics, antinauseants, antineoplastics, antineoplastic adjuvants, antineurtropenics, antiobesity agents, antiobsessionals, antiosteoporotics, antipagentics, antiparkinsonian agents, antiperistaltics, antipheochromocytomas, antipheumocystics, antiprogestins, antiprostatic hypertrophy agents, antiprotozoals, antipuritics, antipsoriatics, antipsychotics, antipyretics, antirickettsials, antiseborrheics, antisepsis agents, antispasmodics, antisyphilitics, antitrombotics, antithrombocythemics, antitubercular agents, antitussives, antiulceratives, antiurolithics, antivenins, antivirals, anxiolytic agents, aromatase inhibitors, or atriopeptidase inhibitors. In some embodiments, one or more implanted agent delivery devices 128 may receive the one or more signals that include information related to one or more agents 162 that include one or more ace-inhibitors, alpha-adrenergic agonists, beta-adrenergic agonists, alpha-adrenergic blockers, beta-adrenergic blockers, adrenocortical steroids, adrenocortical suppressants, adrenocortical hormones, alcohol deterrents, aldose reductase inhibitors, aldosterone antagonists, AMPA receptor antagonists, anabolics, analeptics, analgesics, angrogens, anesthetics, angiotensin II receptor antagonists, anorexics, anthelmintics, antiallergics, antialopecia agents, antiamebics, antiandrogens, antianginals, antiarrhythmics, antiarteriosclerotics, antiarthritics, antirheumatics, antiasthmatics, antibacterials, antibacterial adjuvants, antibiotics, anticholelithogenics, anti cholesteremics, anticholinergics, anticoagulants, anticonvulsants, antidepressants, antidiabetics, antidiarrheals, antidiuretics, antidyskinetics, antieczematics, antiemetics, antiestrogens, antifibrotics, antifungals, antiglaucoma agents, antigonadotropins, antigout agents, antihemophilic factors, antihemorrhagiacs, antihistaminics, antihypercholesterolemics, antihyperlipidemics, antihyperparathyroids, antihyperphosphatemics, antihypertensives, antihyperthyroids, antihypotensives, antihypothyroids, anti-inflammatory agents, antimalarials, antimanics, antimethemoglobinemics, antimigraines, antimuscarinics, antimycotics, antinauseants, antineoplastics, antineoplastic adjuvants, antineurtropenics, antiobesity agents, antiobsessionals, antiosteoporotics, antipagenitics, antiparkinsonian agents, antiperistaltics, antipheochromocytomas, antipheumocystics, antiprogestins, antiprostatic hypertrophy agents, antiprotozoals, antipuritics, antipsoriatics, antipsychotics, antipyretics, antirickettsials, antiseborrheics, antisepsis agents, antispasmodics, antisyphilitics, antithrombotics, antithrombocythemics, antitubercular agents, antitussives, antiulceratives, antiurolithics, antivenins, antivirals, anxiolytic agents, aromatase inhibitors, antiopeptidase inhibitors, or substantially any combination thereof.

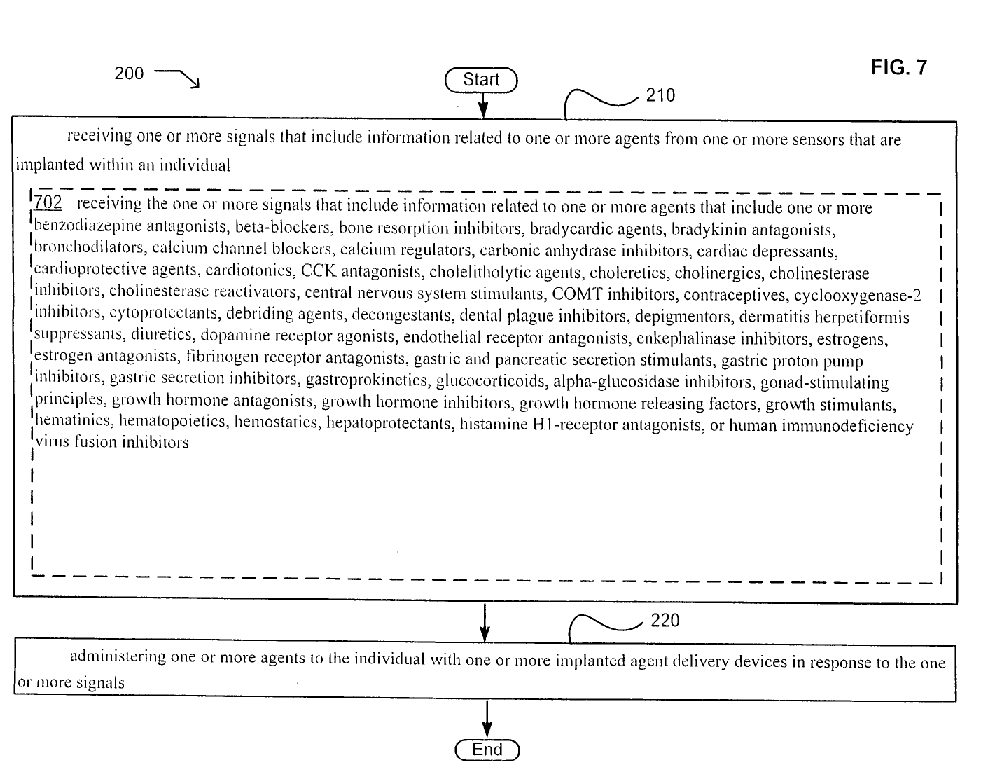

FIG. 7 illustrates alternative embodiments of the example operational flow 200 of FIG. 2. FIG. 7 illustrates example embodiments where the receiving operation 210 may include at least one additional operation. Additional operations may include an operation 702.

At operation 702, the receiving operation 210 may include receiving the one or more signals that include information related to one or more agents that include one or more benzodiazepine antagonists, beta-blockers, bone resorption inhibitors, bradycardic agents, bradykinin antagonists, bronchodilators, calcium channel blockers, calcium regulators, carbonic anhydrase inhibitors, cardiac depressants, cardioprotective agents, cardiotonics, CCK antagonists, cholelitholytic agents, choleretics, cholinergics, cholinesterase inhibitors, cholinesterase reactivators, central nervous system stimulants, COMT inhibitors, contraceptives, cyclooxygenase-2 inhibitors, cytoprotectants, debriding agents, decongestants, dental plague inhibitors, depigmentors, dermatitis herpetiformis suppressants, diuretics, dopamine receptor agonists, endothelial receptor antagonists, enkephalinase inhibitors, estrogens, estrogen antagonists, fibrinogen receptor antagonists, gastric and pancreatic secretion stimulants, gastric proton pump inhibitors, gastric secretion inhibitors, gastroprokinetics, glucocorticoids, alpha-glucosidase inhibitors, gonad-stimulating principles, growth hormone antagonists, growth hormone inhibitors, growth hormone releasing factors, growth stimulants, hematinics, hematopoietics, hemostatics, hepatoprotectants, histamine H1-receptor antagonists, or human immunodeficiency virus fusion inhibitors. In some embodiments, one or more implanted agent delivery devices 128 may receive the one or more signals that include information related to one or more agents that include one or more benzodiazepine antagonists, beta-blockers, bone resorption inhibitors, bradycardic agents, bradykinin antagonists, bronchodilators, calcium channel blockers, calcium regulators, carbonic anhydrase inhibitor-s, cardiac depressants, cardioprotective agents, cardiotonics, CCK antagonists, cholelitholytic agents, choleretics, cholinergics, cholinesterase inhibitors, cholinesterase reactivators, central nervous system stimulants, COMT inhibitors, contraceptives, cyclooxygenase-2 inhibitors, cytoprotectants, debriding agents, decongestants, dental plague inhibitors, depigmentors, dermatitis herpetiformis suppressants, diuretics, dopamine receptor agonists, endothelial receptor antagonists, enkephalinase inhibitors, estrogens, estrogen antagonists, fibrinogen receptor antagonists, gastric and pancreatic secretion stimulants, gastric proton pump inhibitors, gastric secretion inhibitors, gastroprokinetics, glucocorticoids, alpha-glucosidase inhibitors, gonad-stimulating principles, growth hormone antagonists, growth hormone inhibitors, growth hormone releasing factors, growth stimulants, hematinics, hematopoietics, hemostatics, hepatoprotectants, histamine H1-receptor antagonists, human immunodeficiency virus fusion inhibitors, or substantially any combination thereof.

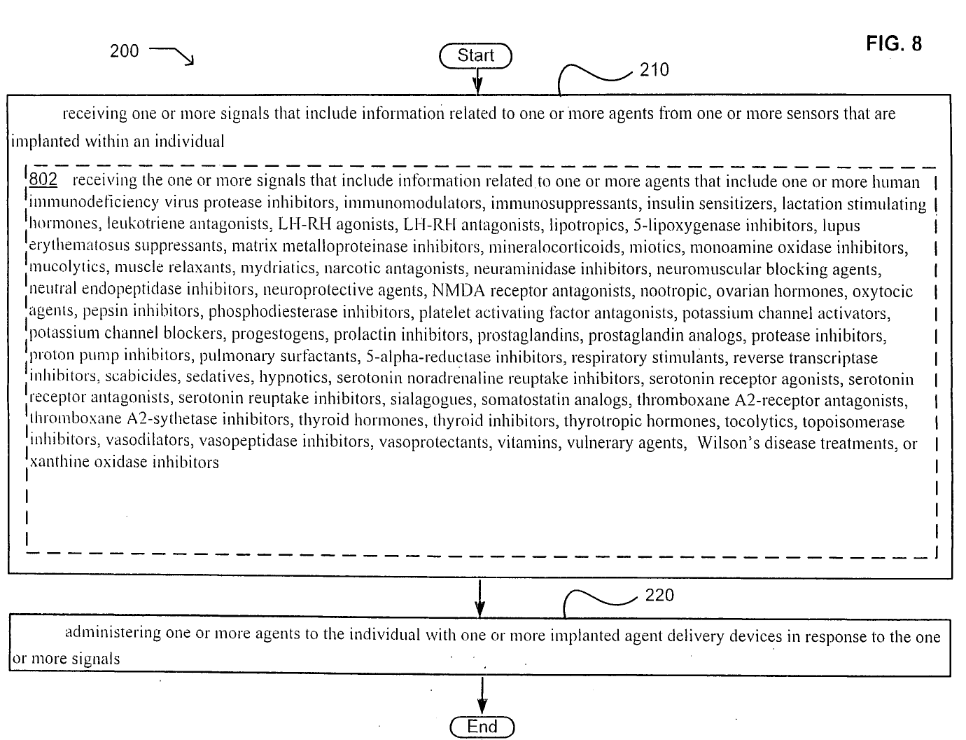

FIG. 8 illustrates alternative embodiments of the example operational flow 200 of FIG. 2. FIG. 8 illustrates example embodiments where the receiving operation 210 may include at least one additional operation. Additional operations may include an operation 802.

At operation 802, the receiving operation 210 may include receiving the one or more signals that include information related to one or more agents that include one or more human immunodeficiency virus protease inhibitors, immunomodulators, immunosuppressants, insulin sensitizers, lactation stimulating hormones, leukotriene antagonists, LH-RH agonists, LH-RH antagonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuraminidase inhibitors, neuromuscular blocking agents, neutral endopeptidase inhibitors, neuroprotective agents, NMDA receptor antagonists, nootropic, ovarian hormones, oxytocic agents, pepsin inhibitors, phosphodiesterase inhibitors, platelet activating factor antagonists, potassium channel activators, potassium channel blockers, progestogens, prolactin inhibitors, prostaglandins, prostaglandin analogs, protease inhibitors, proton pump inhibitors, pulmonary surfactants, 5-alpha-reductase inhibitors, respiratory stimulants, reverse transcriptase inhibitors, scabicides, sedatives, hypnotics, serotonin noradrenaline reuptake inhibitors, serotonin receptor agonists, serotonin receptor antagonists, serotonin reuptake inhibitors, sialagogues, somatostatin analogs, thromboxane A2-receptor antagonists, thromboxane A2-sythetase inhibitors, thyroid hormones, thyroid inhibitors, thyrotropic hormones, tocolytics, topoisomerase inhibitors, vasodilators, vasopeptidase inhibitors, vasoprotectants, vitamins, vulnerary agents, Wilson's disease treatments, or xanthine oxidase inhibitors. In some embodiments, one or more implanted agent delivery devices 128 may receive the one or more signals that include information related to one or more agents that include one or more human immunodeficiency virus protease inhibitors, immunomodulators, immunosuppressants, insulin sensitizers, lactation stimulating hormones, leukotriene antagonists, LH-RH agonists, LH-RH antagonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuraminidase inhibitors, neuromuscular blocking agents, neutral endopeptidase inhibitors, neuroprotective agents, NMDA receptor antagonists, nootropic, ovarian hormones, oxytocic agents, pepsin inhibitors, phosphodiesterase inhibitors, platelet activating factor antagonists, potassium channel activators, potassium channel blockers, progestogens, prolactin inhibitors, prostaglandins, prostaglandin analogs, protease inhibitors, proton pump inhibitors, pulmonary surfactants, 5-alpha-reductase inhibitors, respiratory stimulants, reverse transcriptase inhibitors, scabicides, sedatives, hypnotics, serotonin noradrenaline reuptake inhibitors, serotonin receptor agonists, serotonin receptor antagonists, serotonin reuptake inhibitors, sialagogues, somatostatin analogs, thromboxane A2-receptor antagonists, thromboxane A2-sythetase inhibitors, thyroid hormones, thyroid inhibitors, thyrotropic hormones, tocolytics, topoisomerase inhibitors, vasodilators, vasopeptidase inhibitors, vasoprotectants, vitamins, vulnerary agents, Wilson's disease treatments, xanthine oxidase inhibitors, or substantially any combination thereof.

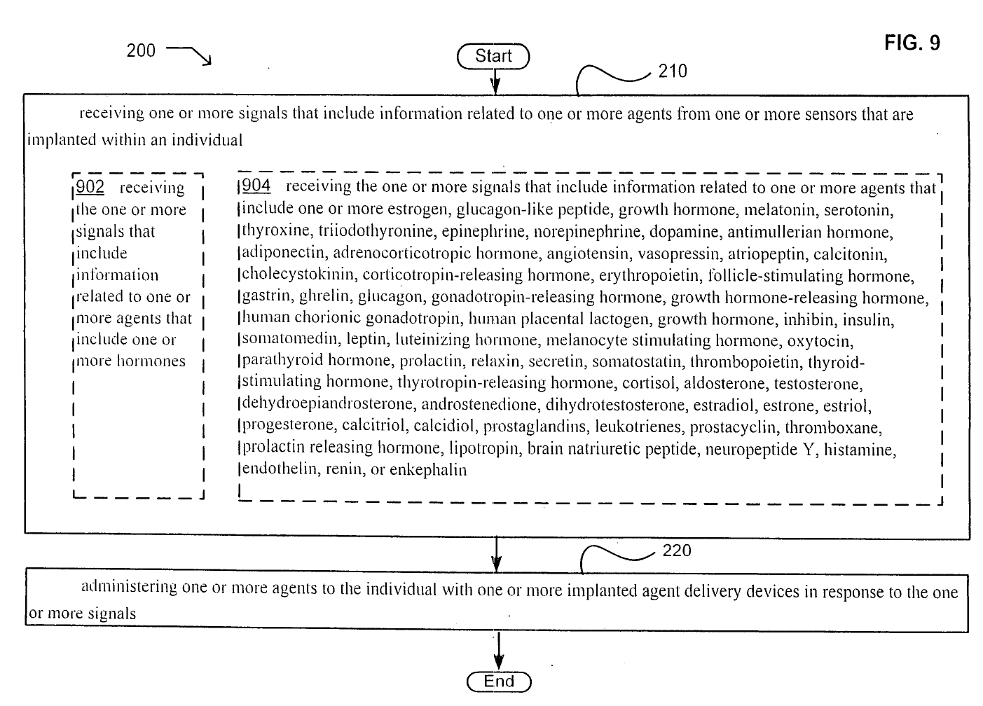

FIG. 9 illustrates alternative embodiments of the example operational flow 200 of FIG. 2. FIG. 9 illustrates example embodiments where the receiving operation 210 may include at least one additional operation. Additional operations may include an operation 902 and/or operation 904.

At operation 902, the receipting operation 210 may include receiving the one or more signals that include information related to one or more agents that include one or more hormones. In some embodiments, one or more implanted agent delivery devices 128 may receive the one or more signals that include information related to one or more agents 162 that include one or more hormones. In some embodiments, one or more implanted agent delivery devices 128 may receive one or more signals that include information related to one or more concentrations of one or more hormones within an individual. In some embodiments, one or more implanted agent deliver), devices 128 may receive one or more signals that include information related to one or more concentrations of one or more hormones within an individual at a single time. In some embodiments, one or more implanted agent delivery devices 128 may receive one or more signals that include information related to one or more concentrations of one or more hormones within an individual at one or more times. In some embodiments, one or more implanted agent delivery devices 128 may receive one or more signals that include information related to one or more changes in the concentration of one or more hormones within an individual.

At operation 904, the receiving operation 210 may include receiving the one or more signals that include information related to one or more agents that include one or more estrogen, glucagon-like peptide, growth hormone, melatonin, serotonin, thyroxine, triiodothyronine, epinephrine, norepinephrine, dopamine, antimullerian hormone, adiponectin, adrenocorticotropic hormone, angiotensin, vasopressin, atriopeptin, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, somatomedin, leptin, luteinizing hormone, melanocyte stimulating hormone, oxytocin, parathyroid hormone, prolactin, relaxin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estradiol, estrone, estriol, progesterone, calcitriol, calcidiol, prostaglandins, leukotrienes, prostacyclin, thromboxane, prolactin releasing hormone, lipotropin, brain natriuretic peptide, neuropeptide A, histamine, endothelin, renin, or enkephalin. In some embodiments, one or more implanted agent delivery devices 128 may receive the one or more signals that include information related to one or more agents 162 that include one or more estrogen, glucagon-like peptide, growth hormone, melatonin, serotonin, thyroxine, triiodothyronine, epinephrine, norepinephrine, dopamine, antimullerian hormone, adiponectin, adrenocorticotropic hormone, angiotensin, vasopressin, atriopeptin, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, somatomedin, leptin, luteinizing hormone, melanocyte stimulating hormone, oxytocin, parathyroid hormone, prolactin, relaxin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estradiol, estrone, estriol, progesterone, calcitriol, calcidiol, prostaglandins, leukotrienes, prostacyclin, thromboxane, prolactin releasing hormone, lipotropin, brain natriuretic peptide, neuropeptide Y, histamine, endothelin, renin, enkephalin, or substantially any combination thereof.

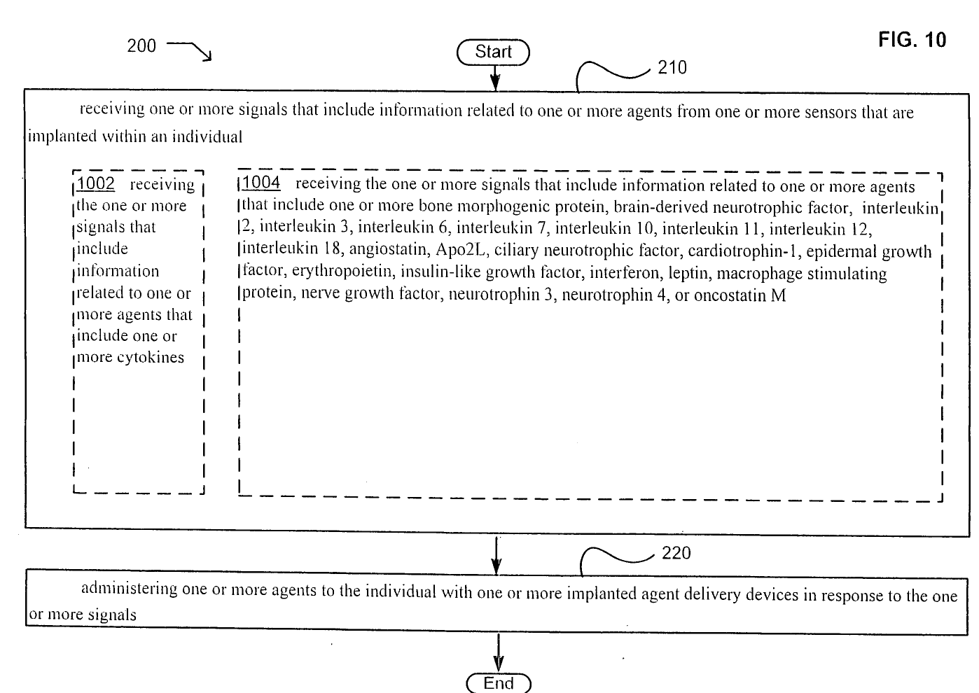

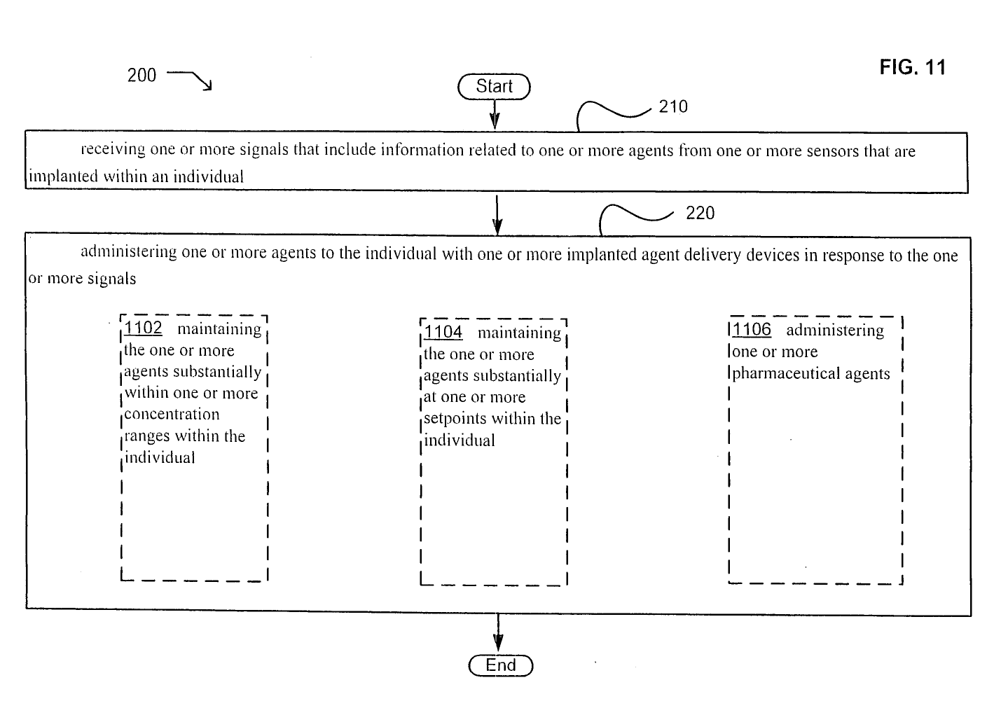

FIG. 10 illustrates alternative embodiments of the example operational flow 200 of FIG. 2. FIG. 10 illustrates example embodiments where the receiving operation 210 may include at least one additional operation. Additional operations may include an operation 1002 and/or operation 1004.

At operation 1002, the receiving operation 210 may include receiving the one or more signals that include information related to one or more agents that include one or more cytokines. In some embodiments, one or more implanted agent delivery devices 128 may receive the one or more signals that include information related to one or more agents 162 that include one or more cytokines. In some embodiments, one or more implanted agent delivery devices 128 may receive one or more signals that include information related to one or more concentrations of one or more cytokines within an individual. In some embodiments, one or more implanted agent delivery devices 128 may receive one or more signals that include information related to one or more concentrations of one or more cytokines lecithin an individual at a single time. In some embodiments, one or more implanted agent delivery devices 128 may receive one or more signals that include information related to one or more concentrations of one or more cytokines within an individual at one or more times. In some embodiments, one or more implanted agent delivery devices 128 may receive one or more signals that include information related to one or more changes in the concentration of one or more cytokines within an individual.

At operation 1004, the receiving operation 910 may include receiving the one or more signals that include information related to one or more agents that include one or more bone morphogenic protein, brain-derived neurotrophic factor, interleukin 2, interleukin 3, interleukin 6, interleukin 7, interleukin 10, interleukin 11, interleukin 12, interleukin 18, angiostatin, Apo2L, ciliary neurotrophic factor, cardiotrophin-1, epidermal growth factor, erythropoietin, insulin-like growth factor, interferon, leptin, macrophage stimulating protein, nerve growth factor, neurotrophin 3, neurotrophin 4, or oncostatin M. In some embodiments, one or more implanted agent delivery devices 128 may receive the one or more signals that include information related to one or more agents 162 that include one or more bone morphogenic protein, brain-derived neurotrophic factor, interleukin 2, interleukin 3, interleukin 6, interleukin 7, interleukin 10, interleukin 11, interleukin 12, interleukin 18, angiostatin, Apo2L, ciliary neurotrophic factor, cardiotrophin-1, epidermal growth factor, erythropoietin, insulin-like growth factor, interferon, leptin, macrophage stimulating protein, nerve growth factor, neurotrophin 3, neurotrophin 4, oncostatin M, or substantially any combination thereof.

FIG. 11 illustrates alternative embodiments of the example operational flow 200 of FIG. 2. FIG. 11 illustrates example embodiments where the administering operation 220 may include at least one additional operation. Additional operations may include an operation 1102, an operation 1104, and/or operation 1106.

At operation 1102, the administering operation 220 may include maintaining the one or more agents substantially within one or more concentration ranges within the individual. In some embodiments, one or more implanted agent delivery devices 128 may maintain the one or more agents 162 substantially within one or more concentration ranges within the individual. In some embodiments, one or more agent delivery devices 128 mall be configured to maintain one or more agents 162 substantially within one or more concentration ranges within an individual. In some embodiments, one or more agent delivery devices 128 may include one or more agent delivery control units 146 that are configured to maintain one or more agents 162 substantially within one or more concentration ranges within an individual. For example, in some embodiments, an agent delivery device 128 may be configured to receive one or more signals from one or more sensors 102 and then administer one or more agents 162 in response to the one or more signals.

In some embodiments, one or more agent delivery devices 128 may receive one or more signals that include information associated with the concentration of one or more agents 162 within an individual. In some embodiments, one or more agent delivery devices 128 may be calibrated to administer an amount of one or more agents 162 to an individual that will cause the concentration of the one or more agents 162 within the individual to reach a concentration that is within one or more concentration ranges. In some embodiments one or more agent delivery devices 128 may be calibrated to administer an amount of one or more agents 162 to an individual that will cause the concentration of the one or more agents 162 within the individual to reach one or more concentrations that are substantially within one or more concentration ranges. For example, in some embodiments, an agent delivery device 128 may include one or more stepper motors 156 that are operably coupled to one or more moveable members 158 that facilitate release of one or more agents 162 from the agent delivery device 128 to facilitate administration of the one or more agents 162 to an individual. In some embodiments, the duration of operation of the one or more stepper motors 156 may be correlated to the amount of one or more agents 162 that are administered to an individual through operation of the agent delivery device 128. Accordingly, in some embodiments, an agent delivery device 128 may receive one or more signals that include information associated with the concentration of an agent 162 within an individual and then operate one or more stepper motors 156 in response to the one or more signals to administer an amount of an agent 162 that causes the concentration of the agent 162 within the individual to be maintained substantially within a concentration range.

In some embodiments, one or more agent delivery devices 128 may receive one or more signals that do not include information that indicates the concentration of one or more detected agents 162. In some embodiments, the one or more signals may be received by one or more agent delivery devices 128 and then processed to determine the concentration of the one or more detected agents 162. In some embodiments, one or more agent deli very devices 128 may then administer an amount of one or more agents 162 to the individual to cause the concentration of the one or more agents 162 within the individual to be substantially maintained within one or more concentration ranges.

In some embodiments, one or more agent delivery devices 128 may receive one or more signals from one or more sensors 102 and administer one or more agents 162 to an individual without determining the concentration of the one or more agents 162 within the individual. For example, in some embodiments, one or more agent delivery devices 128 may administer one or more agents 162 to an individual when the one or more agent delivery devices 128 receive one or more signals that are associated with a value. For example, in some embodiments, one or more agent delivery devices 128 may administer one or more agents 162 to an individual after receiving one or more signals indicating that detection of one or more agents 162 is below a preselected value. In some embodiments, one or more agent delivery devices 128 may stop administering one or more agents 162 to an individual after receiving one or more signals indicating that detection of one or more agents 162 is at and/or above a preselected value. Accordingly, in some embodiments, numerous types of feedback loops between one or more sensors 102 and one or more agent delivery devices 128 may be used to administer one or more agents 162 to an individual.

At operation 1104, the receiving operation 210 may include maintaining the one or more agents substantially at one or more setpoints within the individual. In some embodiments, one or more implanted agent delivery devices 128 may maintain the one or more agents 162 substantially at one or more setpoints within the individual. In some embodiments, one or more agent delivery devices 128 may be configured to maintain one or more agents 162 substantially at one or more setpoints within an individual. In some embodiments, one or more agent delivery devices 128 may include one or more agent delivery control units 146 that are configured to maintain one or more agents 162 substantially, at one or more setpoints within an individual. For example, in some embodiments, an agent delivery device 128 mall be configured to receive one or more signals from one or more sensors 102 and then administer one or more agents 162 in response to the one or more signals.

In some embodiments, one or more agent delivery devices 128 may receive one or more signals that include information associated with the concentration of one or more agents 162 within an individual. In some embodiments, one or more agent delivery devices 128 may be calibrated to administer an amount of one or more agents 162 to an individual that will cause the concentration of the one or more agents 162 within the individual to reach a concentration that is substantially at one or more setpoints within an individual. In some embodiments, one or more agent delivery devices 128 may be calibrated to administer an amount of one or more agents 162 to an individual that will cause the concentration of the one or more agents 162 within the individual to reach one or more concentrations that are substantially at one or more setpoints within an individual. For example, in some embodiments, an agent delivery device 128 may include one or more stepper motors 156 that are operably coupled to one or more moveable members 158 that facilitate release of one or more agents 162 from the agent delivery device 128 to facilitate administration of the one or more agents 162 to an individual. In some embodiments, the duration of operation of the one or more stepper motors 156 may be correlated to the amount of one or more agents 162 that are administered to an individual through operation of the agent delivery device 128. Accordingly, in some embodiments, an agent delivery device 128 may receive one or more signals that include information associated with the concentration of an agent 162 within an individual and then operate one or more stepper motors 156 in response to the one or more signals to administer an amount of an agent 162 that causes the concentration of the agent 162 within the individual to be maintained substantially at one or more setpoints within an individual.

In some embodiments, one or more agent delivery devices 128 may receive one or more signals that do not include information that indicates the concentration of one or more detected agents 162. In some embodiments, the one or more signals may be received by one or more agent delivery devices 128 and then processed to determine the concentration of the one or more detected agents 162. In some embodiments one or more agent delivery devices 128 may then administer an amount of one or more agents 162 to the individual to cause the concentration of the one or more agents 16′ within the individual to be substantially maintained at one or more setpoints within an individual.

In some embodiments, one or more agent delivery devices 128 may receive one or more signals from one or more sensors 102 and administer one or more agents 162 to an individual without determining the concentration of the one or more agents 162 within the individual. For example, in some embodiments, one or more agent delivery devices 128 may administer one or more agents 162 to an individual when the one or more agent delivery devices 128 receive one or more signals that are associated with a value. For example, in some embodiments, one or more agent delivery devices 128 may administer one or more agents 162 to an individual after receiving one or more signals indicating that detection of one or more agents 162 is below a preselected setpoint. In some embodiments, one or more agent delivery devices 128 may stop administering one or more agents 16 to an individual after receiving one or more signals indicating that detection of one or more agents 162 is at and/or above a preselected setpoint. Accordingly, in some embodiments, numerous types of feedback loops between one or more sensors 102 and one or more agent delivery devices 128 may be used to administer one or more agents 162 to an individual.

At operation 1106, the receiving operation 210 may include administering one or more pharmaceutical agents. In some embodiments, one or more implanted agent delivery devices 128 may administer one or more pharmaceutical agents 162. One or more agent deliver), devices 128 may administer numerous types of pharmaceutical agents 162 to an individual. In some embodiments, one or more pharmaceutical agents 162 may be administered that are substantially immediately available to the individual. In some embodiments, one or more pharmaceutical agents 162 that are formulated for sustained release may be administered to an individual. For example, in some embodiments, one or more agent delivery devices 128 may be configured to administer one or more pharmaceutical agents 162 to an individual on a daily basis. Accordingly, in some embodiments, one or more pharmaceutical agents 162 may be formulated for administration to an individual in accordance with the frequency with which the one or more pharmaceutical agents 162 are to be administered to an individual. For example, in some embodiments, one or more pharmaceutical agents 106 may be formulated for release other a four hour period upon administration if they are to be administered to an individual six times per twenty four hour period.

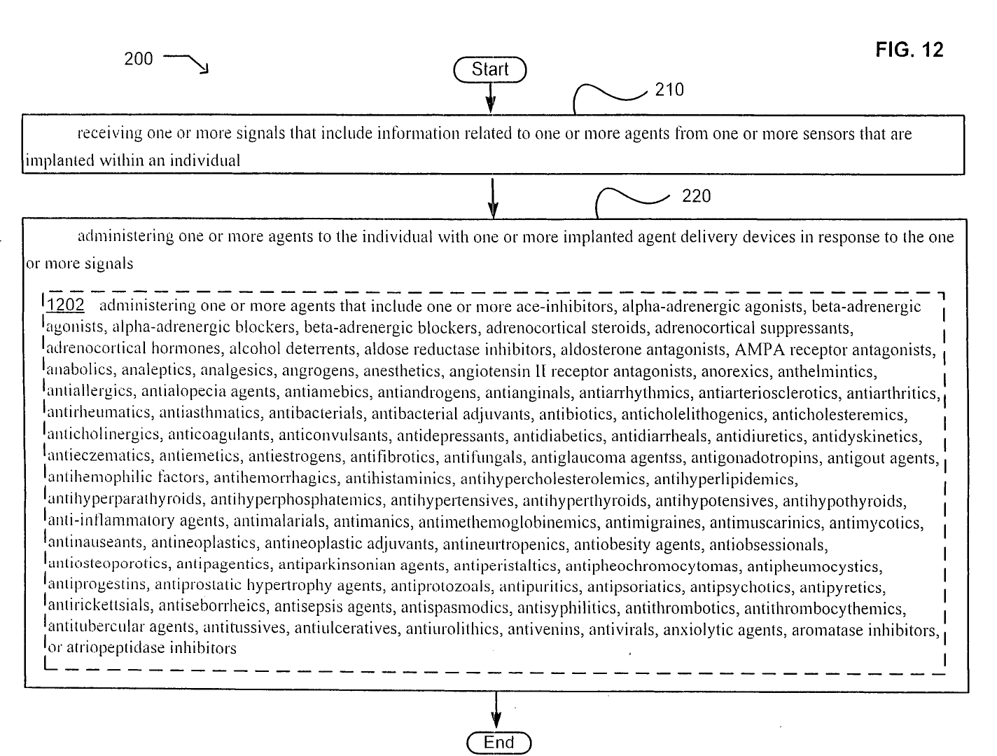

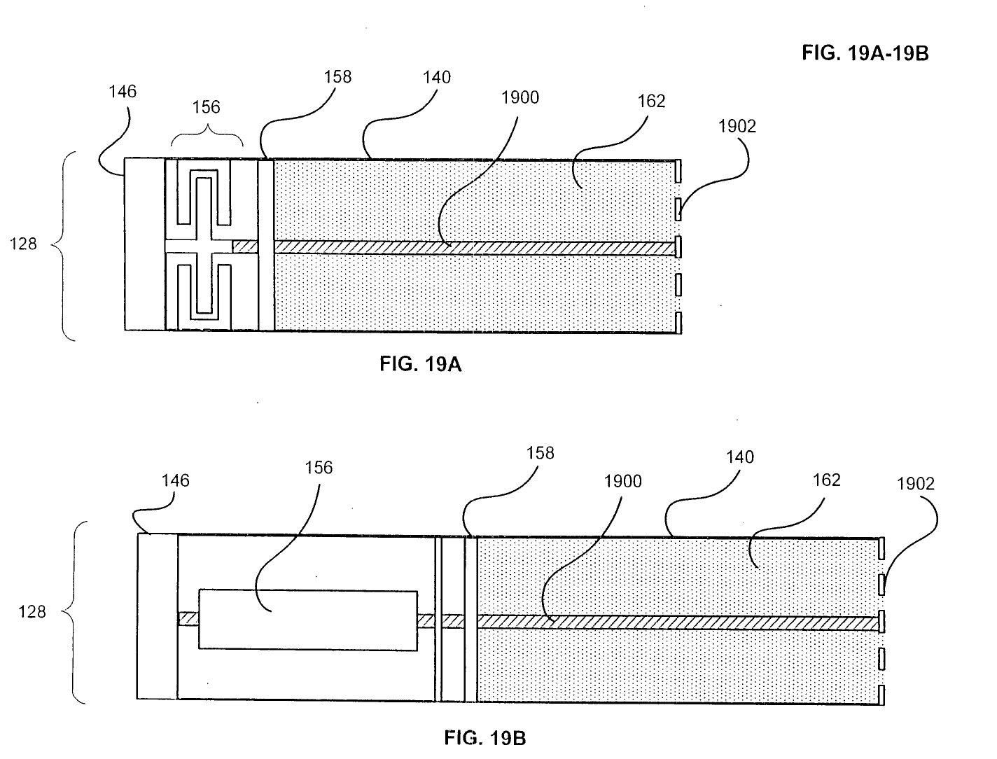

FIG. 12 illustrates alternative embodiments of the example operational flow 200 of FIG. 2. FIG. 19 illustrates example embodiments where the administering operation 220 may include at least one additional operation. Additional operations may include an operation 1202.

At operation 1202, the administering operation 220 may include administering one or more agents that include one or more ace-inhibitors, alpha-adrenergic agonists, beta-adrenergic agonists, alpha-adrenergic blockers, beta-adrenergic blockers, adrenocortical steroids, adrenocortical suppressants, adrenocortical hormones, alcohol deterrents, aldose reductase inhibitors, aldosterone antagonists, AMPA receptor antagonists, anabolics, analeptics, analgesics, angrogens, anesthetics, angiotensin II receptor antagonists, anorexics, anthelmintics, antiallergics, antialopecia agents, antiamebics, antiandrogens, antianginals, antiarrhythmics, antiarteriosclerotics, antiarthritics, antirheumatics, antiasthmatics, antibacterials, antibacterial adjuvants, antibiotics, anticholelithogenics, anticholesteremics, anticholinergics, anticoagulants, anticonvulsants, antidepressants, antidiabetics, antidiarrheals, antidiuretics, antidyskinetics, antieczematics, antiemetics, antiestrogens, antifibrotics, antifungals, antiglaucoma agentss, antigonadotropins, antigout agents, antihemophilic factors, antihemorrhagics, antihistaminics, antihypercholoesterolemics, antihyperlipidemics, antihyperparathyroids, antihyperphosphatemics, antihypertensives, antihyperthyroids, antihypotensives, antihypothyroids, anti-inflammatory agents, antimalarials, antimanics, antimethemoglobinemics, antimigraines, antimuscarinics, antimycotics, antinauseants, antineoplastics, antineoplastic adjuvants, antineurtropenics, antiobesity agents, antiobsessionals, antiosteoporotics, antipagentics, antiparkinsonian agents, antiperistaltics, antipheochromocytomas, antipheumocystics, antiprogestins, antiprostatic hypertrophy agents, antiprotozoals, antipuritics, antipsoriatics, antipsychotics, antipyretics, antirickettsials, antiseborrheics, antisepsis agents, antispasmodics, antisyphilitics, antithrombotics, antithrombocythemics, antitubercular agents, antitussives antiulceratives, antiurolithics, antivenins, antivirals, anxiolytic agents, aromatase inhibitors, or atriopeptidase inhibitors. In some embodiments, one or more implanted agent delivery devices 128 may administer one or more agents 162 that include one or more ace-inhibitors, alpha-adrenergic agonists, beta-adrenergic agonists, alpha-adrenergic blockers, beta-adrenergic blockers, adrenocortical steroids, adrenocortical suppressants, adrenocortical hormones alcohol deterrents, aldose reductase inhibitors, aldosterone antagonists, AMPA receptor antagonists, anabolics, analeptics, analgesics, angrogens, anesthetics, angiotensin II receptor antagonists, anorexics, anthelmintics, antiallergics, antialopecia agents, antiamebics, antiandrogens, antianginals, antiarrhythmics, antiarteriosclerotics, antiarthritics, antirheumatics, antiasthmatics, antibacterials, antibacterial adjuvants, antibiotics, anticholelithogenics, anti cholesteremics, anticholinergics, anticoagulants, anti convulsants, antidepressants, antidiabetics, antidiarrheals, antidiuretics, antidyskinetics, antieczematics, antiemetics, antiestrogens, antifibrotics, antifungals, antiglaucoma agentss, antigonadotropins, antigout agents, antihemophilic factors, antihemorrhagics, antihistaminics, antihypercholesterolemics, antihyperlipidemics, antihyperparathyroids, antihyperphosphatemics, antihypertensives, antihyperthyroids, antihypotensives, antihypothyroids, anti-inflammatory agents, antimalarials, antimanics, antimethemoglobinemics, antimigraines, antimuscarinics, antimycotics, antinauseants, antineoplastics, antineoplastic adjuvants, antineurtropenics, antiobesity agents, antiobsessionals, antiosteoporotics, antipagentics, antiparkinsonian agents, antiperistaltics, antipheochromocytomas, antipheumocystics, antiprogestins, antiprostatic hypertrophy agents, antiprotozoals, antipuritics, antipsoriatics, antipsychotics, antipyretics, antirickettsials, antiseborrheics, antisepsis agents, antispasmodics, antisyphilitics, antithrombotics, antithrombocythemics, antitubercular agents, antitussives, antiulceratives, antiurolithics, antivenins, antivirals, anxiolytic agents, aromatase inhibitors, atriopeptidase inhibitors, or substantially any combination thereof.

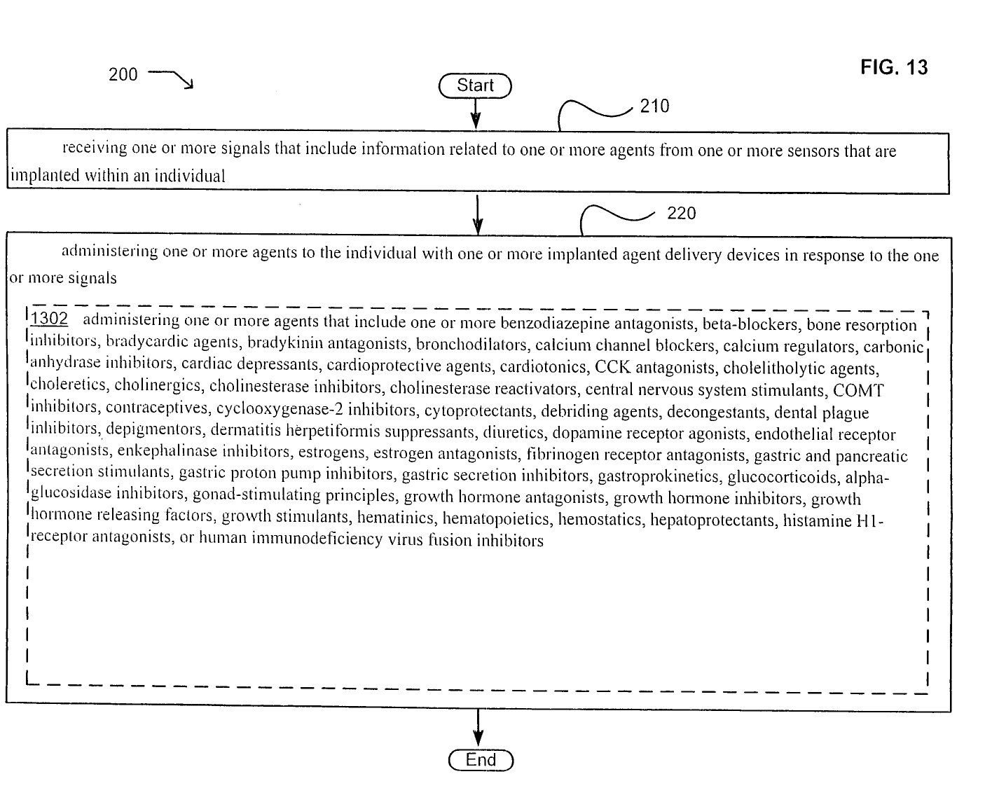

FIG. 13 illustrates alternative embodiments of the example operational flow 200 of FIG. 2. FIG. 13 illustrates example embodiments where the administering operation 220 may include at least one additional operation. Additional operations may include an operation 1302.

At operation 1320, the administering operation 220 may include administering one or more agents that include one or more benzodiazepine antagonists, beta-blockers, bone resorption inhibitors, bradycardic agents, bradykinin antagonists, bronchodilators, calcium channel blockers. Calcium regulators, carbonic anhydrase inhibitors, cardiac depressants, cardioprotective agents, cardiotonics, CCK antagonists, cholelitholytic agents, choleretics, cholinergics cholinesterase inhibitors, cholinesterase reactivators, central nervous system stimulants, COMT inhibitors, contraceptives, cyclooxygenase-2 inhibitors, cytoprotectants, debriding agents, decongestants, dental plague inhibitors, depigmentors, dermatitis herpetiformis suppressants, diuretics, dopamine receptor agonists, endothelial receptor antagonists, enkephalinase inhibitors, estrogens, estrogen antagonists, fibrinogen receptor antagonists, gastric and pancreatic secretion stimulants, gastric proton pump inhibitors, gastric secretion inhibitors, gastroprokinetics, glucocorticoids, alpha-glucosidase inhibitors, gonad-stimulating principles, growth hormone antagonists, growth hormone inhibitors, growth hormone releasing factors, growth stimulants, hematinics, hematopoietics, hemostatics, hepatoprotectants, histamine H1-receptor antagonists, or human immunodeficiency virus fusion inhibitors. In some embodiments, one or more implanted agent delivery devices 128 may administer one or more agents that include one or more benzodiazepine antagonists, beta-blockers, bone resorption inhibitors, bradycardic agents, bradykinin antagonists, bronchodilators, calcium channel blockers, calcium regulators, carbonic anhydrase inhibitors, cardiac depressants, cardioprotective agents, cardiotonics, CCK antagonists, cholelitholytic agents, choleretics, cholinergics, cholinesterase inhibitors, cholinesterase reactivators, central nervous system stimulants, COMT inhibitors, contraceptives, cyclooxygenase-2 inhibitors, cytoprotectants, debriding agents, decongestants, dental plague inhibitors, depigmentors, dermatitis herpetiformis suppressants, diuretics, dopamine receptor agonists, endothelial receptor antagonists, enkephalinase inhibitors, estrogens, estrogen antagonists, fibrinogen receptor antagonists, gastric and pancreatic secretion stimulants, gastric proton pump inhibitors, gastric secretion inhibitors, gastroprokinetics, glucocorticoids, alpha-glucosidase inhibitors, gonad-stimulating principles, growth hormone antagonists, growth hormone inhibitors, growth hormone releasing factors, growth stimulants, hematinics, hematopoietics, hemostatics, hepatoprotectants, histamine H1-receptor antagonists, human immunodeficiency virus fusion inhibitors, or substantially, any, combination thereof.

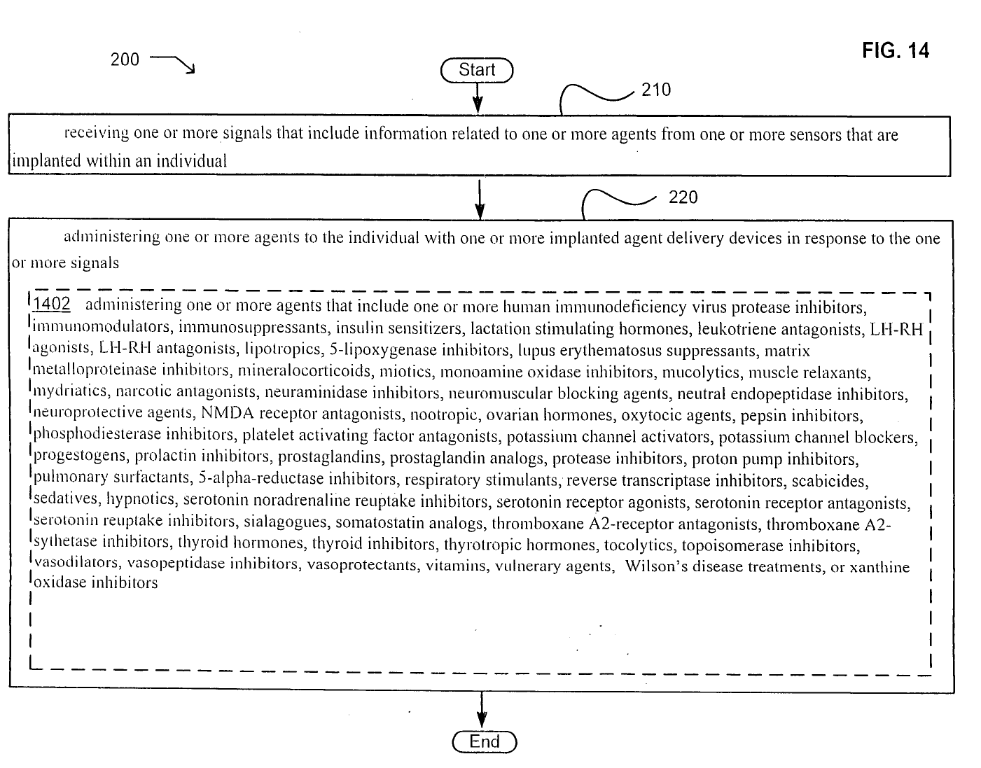

FIG. 14 illustrates alternative embodiments of the example operational flow, 200 of FIG. 2. FIG. 14 illustrates example embodiments where the administering operation 220 may include at least one additional operation. Additional operations malt include an operation 1402.

At operation 1402, the administering operation 220 may include administering one or more agents that include one or more human immunodeficiency virus protease inhibitors, immunomodulators, immunosuppressants, insulin sensitizers, lactation stimulating hormones, leukotriene antagonists, LH-RH agonists, LH-RH antagonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuraminidase inhibitors, neuromuscular blocking agents, neutral endopeptidase inhibitors, neuroprotective agents, NMDA receptor antagonists, nootropic, ovarian hormones, oxytocic agents, pepsin inhibitors, phosphodiesterase inhibitors, platelet activating factor antagonists, potassium channel activators, potassium channel blockers, progestogens, prolactin inhibitors, prostaglandins, prostaglandin analogs, protease inhibitors, proton pump inhibitors, pulmonary surfactants, 5-alpha-reductase inhibitors, respiratory stimulants, reverse transcriptase inhibitors, scabicides, sedatives, hypnotics, serotonin noradrenaline reuptake inhibitors, serotonin receptor agonists, serotonin receptor antagonists, serotonin reuptake inhibitors, sialagogues, somatostatin analogs, thromboxane A2-receptor antagonists, thromboxane A2-sythetase inhibitors, thyroid hormones, thyroid inhibitors, thyrotropic hormones, tocolytics, topoisomerase inhibitors, vasodilators, vasopeptidase inhibitors, vasoprotectants, vitamins, vulnerary agents, Wilson's disease treatments, or xanthine oxidase inhibitors. In some embodiments, one or more implanted agent delivery devices 128 may administer one or more agents 162 that include one or more human immunodeficiency virus protease inhibitors, immunomodulators, immunosuppressants, insulin sensitizers, lactation stimulating hormones, leukotriene antagonists, LH-RH agonists, LH-RH antagonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuraminidase inhibitors, neuromuscular blocking agents, neutral endopeptidase inhibitors, neuroprotective agents, NMDA receptor antagonists, nootropic, ovarian hormones, oxytocic agents, pepsin inhibitors, phosphodiesterase inhibitors, platelet activating factor antagonists, potassium channel activators, potassium channel blockers, progestogens, prolactin inhibitors, prostaglandins, prostaglandin analogs, protease inhibitors, proton pump inhibitors, pulmonary surfactants, 5-alpha-reductase inhibitors, respiratory stimulants, reverse transcriptase inhibitors, scabicides, sedatives, hypnotics, serotonin noradrenaline reuptake inhibitors, serotonin receptor agonists, serotonin receptor antagonists, serotonin reuptake inhibitors, sialagogues, somatostatin analogs, thromboxane A2-receptor antagonists, thromboxane A2-sythetase inhibitors, thyroid hormones, thyroid inhibitors, thyrotropic hormones, tocolytics, topoisomerase inhibitors, vasodilators, vasopeptidase inhibitors, vasoprotectants, vitamins, vulnerary agents, Wilson's disease treatments, xanthine oxidase inhibitors, or substantially any combination thereof.

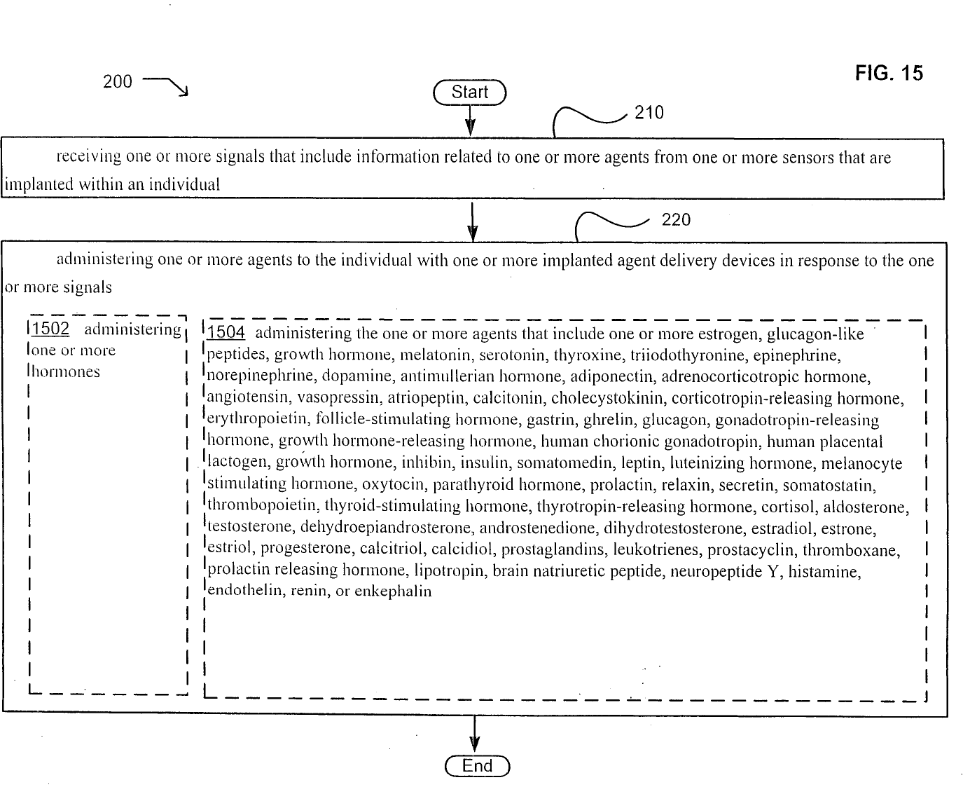

FIG. 15 illustrates alternative embodiments of the example operational flow 200 of FIG. 2. FIG. 15 illustrates example embodiments where the administering operation 220 may include at least one additional operation. Additional operations may include an operation 1502 and/or operation 1504.

At operation 1509, the administering operation 220 may include administering one or more hormones. In some embodiments, one or more implanted agent delivery devices 128 may administer one or more hormones. One or more agent delivery devices 128 may administer numerous types of hormones to an individual. In some embodiments, one or more hormones may be administered that are substantially immediately available to the individual. In some embodiments, one or more hormones that are formulated for sustained release may be administered to an individual. For example, in some embodiments, one or more agent delivery devices 128 may be configured to administer one or more hormones to an individual on a daily basis. Accordingly, in some embodiments, one or more hormones may be formulated for administration to an individual in accordance with the frequency with which the one or more hormones are to be administered to an individual. For example, in some embodiments, one or more hormones ma), be formulated for release over a four hour period upon administration if they are to be administered to an individual six times per twenty four hour period.

At operation 1504, the administering operation 220 may, include administering the one or more agents that include one or more estrogen, glucagon-like peptides, growth hormone, melatonin, serotonin, thyroxine, triiodothyronine, epinephrine, norepinephrine, dopamine, antimullerian hormone, adiponectin, adrenocorticotropic hormone, angiotensin, vasopressin, atriopeptin, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, somatomedin, leptin, luteinizing hormone, melanocyte stimulating hormone, oxytocin, parathyroid hormone, prolactin, relaxin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estradiol, estrone, estriol, progesterone, calcitriol, calcidiol, prostaglandins, leukotrienes, prostacyclin, thromboxane, prolactin releasing hormone, lipotropin, brain natriuretic peptide, neuropeptide Y, histamine, endothelin, renin, or enkephalin. In some embodiments, one or more implanted agent delivery devices 128 may administer one or more agents 162 that include one or more estrogen, glucagon-like peptides, growth hormone, melatonin, serotonin, thyroxine, triiodothyronine, epinephrine, norepinephrine, dopamine, antimullerian hormone, adiponectin, adrenocorticotropic hormone, angiotensin, vasopressin, atriopeptin, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, somatomedin, leptin, luteinizing hormone, melanocyte stimulating hormone, oxytocin, parathyroid hormone, prolactin, relaxin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estradiol, estrone, estriol, progesterone, calcitriol, calcidiol, prostaglandins, leukotrienes, prostacyclin, thromboxane, prolactin releasing hormone, lipotropin, brain natriuretic peptide, neuropeptide Y, histamine, endothelin, rein, enkephalin, or substantially any combination thereof.

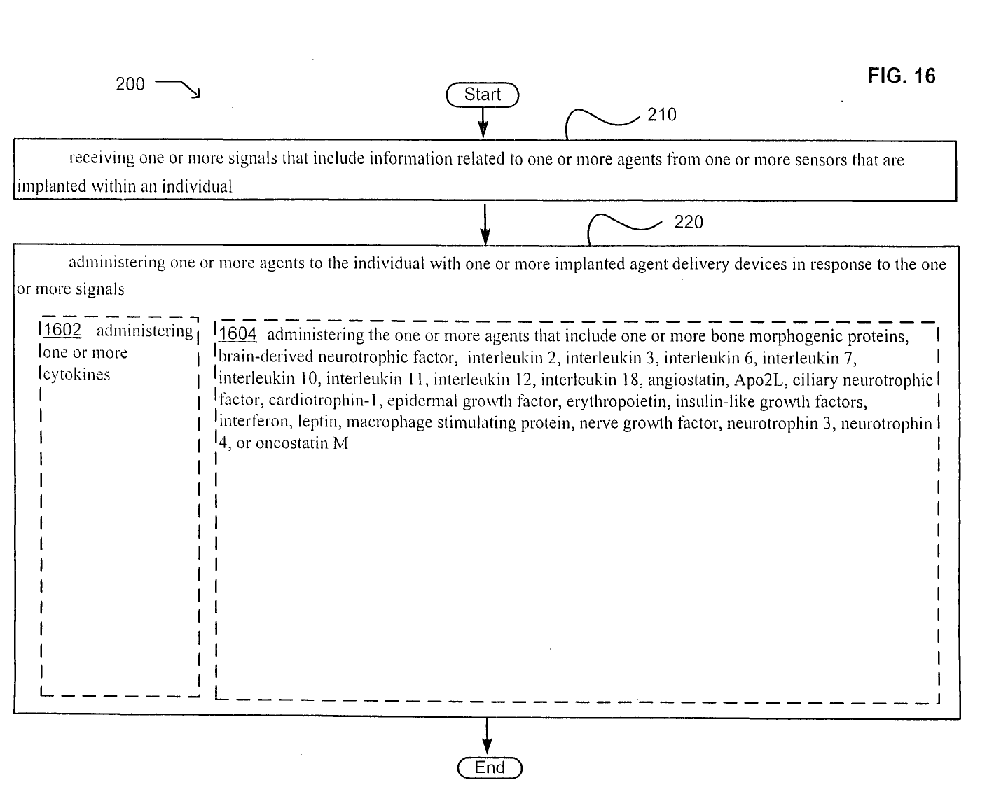

FIG. 16 illustrates alternative embodiments of the example operational flow 200 of FIG. 2. FIG. 16 illustrates example embodiments where the administering operation 220 may include at least one additional operation. Additional operations mats include an operation 1602 and/or operation 1604.

At operation 1609, the administering operation 220 may include administering one or more cytokines. In some embodiments, one or more implanted agent delivery devices 128 may administer one or more cytokines. One or more agent delivery devices 128 may administer numerous types of cytokines to an individual. In some embodiments, one or more cytokines may be administered that are substantially immediately available to the individual. In some embodiments, one or more cytokines that are formulated for sustained release may be administered to an individual. For example, in some embodiments, one or more agent delivery devices 128 may be configured to administer one or more cytokines to an individual on a daily basis. Accordingly, in some embodiments, one or more cytokines may be formulated for administration to an individual in accordance with the frequency with which the one or more cytokines are to be administered to an individual. For example, in some embodiments, one or more cytokines may be formulated for release over a four hour period upon administration if they are to be administered to an individual six times per twenty four hour period.

At operation 1604, the administering operation 220 may include administering the one or more agents that include one or more bone morphogenic proteins, brain-derived neurotrophic factor, interleukin 2, interleukin 3, interleukin 6, interleukin 7, interleukin 10, interleukin 11, interleukin 12, interleukin 18, angiostatin, Apo2L, ciliary neurotrophic factor, cardiotrophin-1, epidermal growth factor, erythropoietin, insulin-like growth factors, interferon, leptin, macrophage stimulating protein, nerve growth factor, neurotrophin 3, neurotrophin 4, or oncostatin M. In some embodiments, one or more implanted agent delivery devices 128 may administer one or more agents 162 that include one or more bone morphogenic proteins, brain-derived neurotrophic factor, interleukin 2, interleukin 3, interleukin 6, interleukin 7, interleukin 10, interleukin 11, interleukin 12, interleukin 18, angiostatin, Apo2L, ciliary neurotrophic factor, cardiotrophin-1, epidermal growth factor, erythropoietin, insulin-like growth factors, interferon, leptin, macrophage stimulating protein, nerve growth factor, neurotrophin 3, neurotrophin 4, oncostatin M, or substantially any combination thereof.

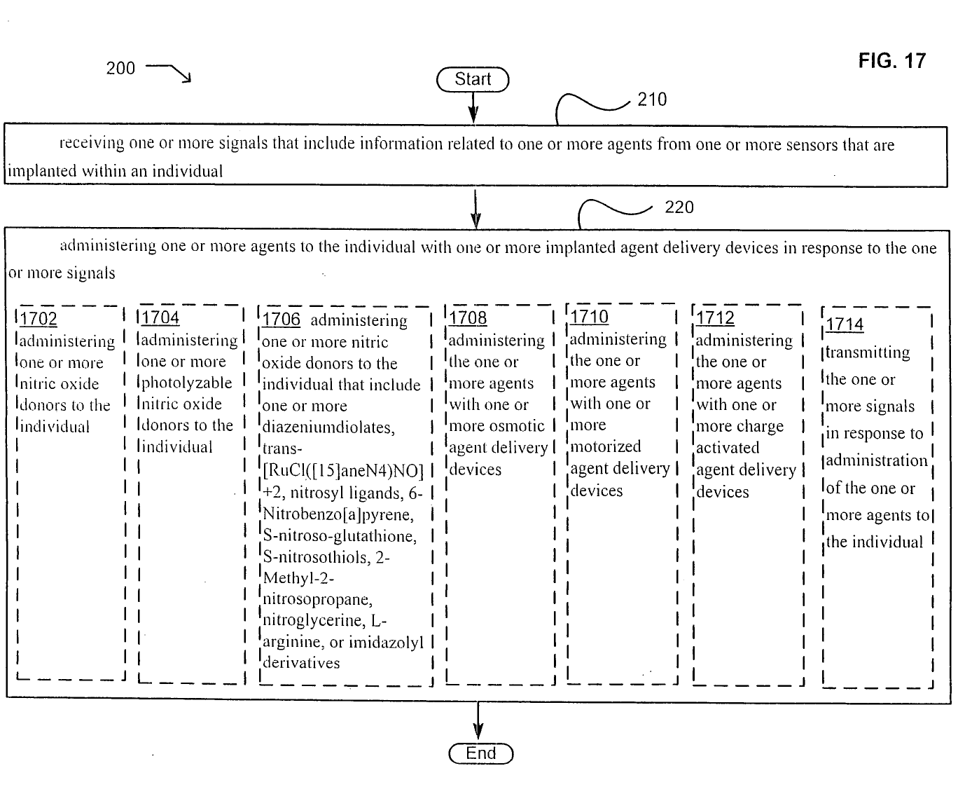

FIG. 17 illustrates alternative embodiments of the example operational flow 200 of FIG. 2. FIG. 17 illustrates example embodiments where the administering operation 220 may include at least one additional operation. Additional operations may include an operation 1702, an operation 1704, an operation 1706, an operation 1708, an operation 1710, an operation 1719, and/or operation 1714.

At operation 1702, the administering operation 220 may include administering one or more nitric oxide donors to the individual. In some embodiments, one or more implanted agent delivery devices 128 may administer one or more nitric oxide donors to the individual. In some embodiments, one or more agent delivery devices 128 may be configured to deliver one or more nitric oxide donors to the genital region of an individual. Accordingly, in some embodiments, one or more agent delivery devices 128 may be used to administer one or more nitric oxide donors to an individual to reduce the effects of sexual dysfunction (e.g., U.S. Pat. No. 6,290,981). In some embodiments, one or more agent delivery devices 128 may be configured to administer one or more nitric oxide donors to the vascular system of an individual. Accordingly, in some embodiments, one or more agent delivery devices 128 may be used to administer one or more nitric oxide donors to an individual to reduce the effects of a stroke (e.g., U.S. Pat. No. 5,385,940).

At operation 1704, the administering operation 220 may include administering one or more photolyzable nitric oxide donors to the individual. In some embodiments, one or more implanted agent delivery devices 128 may administer one or more photolyzable nitric oxide donors to the individual. In some embodiments, one or more agent delivery devices 128 may be configured to deliver one or more photolyzable nitric oxide donors to the genital region of an individual. Accordingly, in some embodiments, one or more agent delivery devices 128 may be used to administer one or more photolyzable nitric oxide donors to an individual to reduce the effects of sexual dysfunction (e.g., U.S. Pat. No. 6,290,981). In some embodiments, one or more agent delivery devices 128 may, be configured to administer one or more photolyzable nitric oxide donors to the vascular system of an individual. Accordingly in some embodiments, one or more agent delivery devices 128 may be used to administer one or more photolyzable nitric oxide donors to an individual to reduce the effects of a stroke (e.g., U.S. Pat. No. 5,385,940).

At operation 1706, the administering operation 290 may include administering one or more nitric oxide donors to the individual that include one or more diazeniumdiolates, trans-[RuCl([15]aneN4)NO]+2, nitrosyl ligands, 6-Nitrobenzo[a]pyrene, S-nitroso-glutathione, S-nitrosothiols, 2-Methyl-2-nitrosopropane, nitroglycerine, L-arginine, or imidazolyl derivatives. In some embodiments, one or more implanted agent delivery devices 128 may administer one or more nitric oxide donors to the individual that include one or more diazeniumdiolates, trans-[RuCl([15]aneN4)NO]+2, nitrosyl ligands, 6-Nitrobenzo[a]pyrene, S-nitroso-glutathione, S-nitrosothiols, 2-Methyl-2-nitrosopropane, nitroglycerine, L-arginine, imidazolyl derivatives, or substantially any combination thereof.

At operation 1708, the administering operation 220 may include administering the one or more agents with one or more osmotic agent delivery devices. In some embodiments, one or more implanted agent delivery devices 128 may administer one or more agents 162 with one or more osmotic agent delivery devices 128. In some embodiments, one or more osmotic delivery devices may include one or more controllable entry ports. In some embodiments, one or more osmotic delivery devices may include one or more controllable exit ports. In some embodiments, one or more osmotic delivery devices may include one or more controllable-entry ports and one or more controllable exit ports. In some embodiments, one or more osmotic delivery devices may include one or more electromagnetically controlled entry ports. In some embodiments, one or more osmotic delivery devices may include one or more electromagnetically controlled exit ports. In some embodiments, one or more osmotic delivery devices may include one or more electromagnetically controlled entry ports and one or more electromagnetically controlled exit ports. Accordingly, in some embodiments, entry of fluid into one or more osmotic delivery devices may be controlled through regulation of the electromagnetically controlled entry ports. In some embodiments, exit of one or more agents 162 from one or more osmotic deliver y devices mail be controlled through regulation of the electromagnetically controlled exit ports. In some embodiments, entry of fluid into one or more osmotic delivery devices and exit of one or more agents 162 from one or more osmotic delivery devices may be controlled through regulation of the electromagnetically controlled entry and exit ports. In some embodiments, one or more osmotic delivery devices may include one or more entry ports that include one or more shape memory materials (e.g., U.S. Pat. No. 6,454,759). In some embodiments, one or more osmotic delivery devices may include one or more exit ports that include one or more shape memory materials (e.g., U.S. Pat. No. 6,454,759). In some embodiments, one or more osmotic delivery devices may include one or more entry ports and one or more exit ports that include one or more shape memory materials (e.g., U.S. Pat. No. 6,454,759). Accordingly, in some embodiments, entry of fluid into one or more osmotic delivery devices may be controlled through regulation of the shape memory polymer. In some embodiments, exit of one or more agents 162 from one or more osmotic delivery devices may be controlled through regulation of the shape memory polymer. In some embodiments, entry of fluid into one or more osmotic delivery devices and exit of one or more agents 162 from one or more osmotic delivery devices may be controlled through regulation of the shape memory polymer.

At operation 1710, the administering operation 220 may include administering the one or more agents with one or more motorized agent delivery devices. In some embodiments, one or more implanted agent delivery devices 128 may administer one or more agents 162 with one or more motorized agent delivery devices 128. Numerous types of motors 156 may be associated with one or more agent delivery devices 128. Examples of such motors 156 include, but are not limited to, stepper motors 156, squiggle motors 156, acoustic motors 156, piezoelectric motors 156, and the like (e.g., Spanner, Survey of the Various Operating Principles of Ultrasonic Piezomotors, White Pater for ACTUATOR 2006; New Scale Technologies, Inc., Victor, N.Y.; PI (Physik Instrumente) L.P., Auburn, Mass.; U.S. Pat. No. 7,314,443). In some embodiments, a motor 156 may be operably coupled to a moveable member 158 so that operation of the motor 156 causes movement of the moveable member 158. In some embodiments, a motor 156 may be operably coupled to a moveable member 158 with a threaded member such that rotation of the threaded member facilitates movement of the moveable member 158. Accordingly, in some embodiments, one or more motors 156 may be rotary motors 156. In some embodiments, a motor 156 may be operably coupled to a moveable member 158 with a ratcheted member such that motion of the ratcheted member facilitates movement of the moveable member 158. Accordingly, in some embodiments, one or more motors 156 may be linear motors 156.

At operation 1712, the administering operation 220 may include administering the one or, more agents with one or more charge activated agent delivery devices. In some embodiments, one or more implanted agent delivery devices 128 may administer the one or more agents 162 with one or more charge activated agent delivery devices 128. For example, in some embodiments, one or more agent delivery devices 128 may include one or more compartments that enclose one or more agents 162 that are configured to administer one or more agents 162 upon application of an electric field (e.g., Massachusetts Institute of Technology (2008, Feb. 14). Thin-film ‘Micro Pharmacy’ Can Be Implanted To Deliver Controlled Drug Doses To Targeted Locations. Science Daily). In some embodiments, one or more agent delivery devices 128 may include one or more agent reservoirs 142 that are covered with a sacrificial layer (e.g., gold foil) that may be unmasked through the application of an electrical current (e.g., Pan et al., Proceedings of the 26^{th} Annual International Conference of the IEEE EMBS, San Francisco, Calif., USA, Sep. 1-5, 2004). In some embodiments, one or more agent delivery devices 128 may include one or more agent reservoirs 142 that are covered with a shape memory material that may be unmasked through the application of an electrical current (e.g., U.S. Pat. No. 6,454,759).

At operation 1714, the administering operation 290 may include transmitting the one or more signals in response to administration of the one or more agents to the individual. In some embodiments, one or more implanted agent delivery devices 128 may transmit one or more signals in response to administration of the one or more agents 162 to the individual. For example, in some embodiments, one or more agent delivery devices 128 may transmit one or more signals to an external interface 168 following administration of one or more agents 162. In some embodiments, one or more agent delivery devices 128 may transmit one or more signals to one or more sensors 102 following, administration of one or more agents 162.

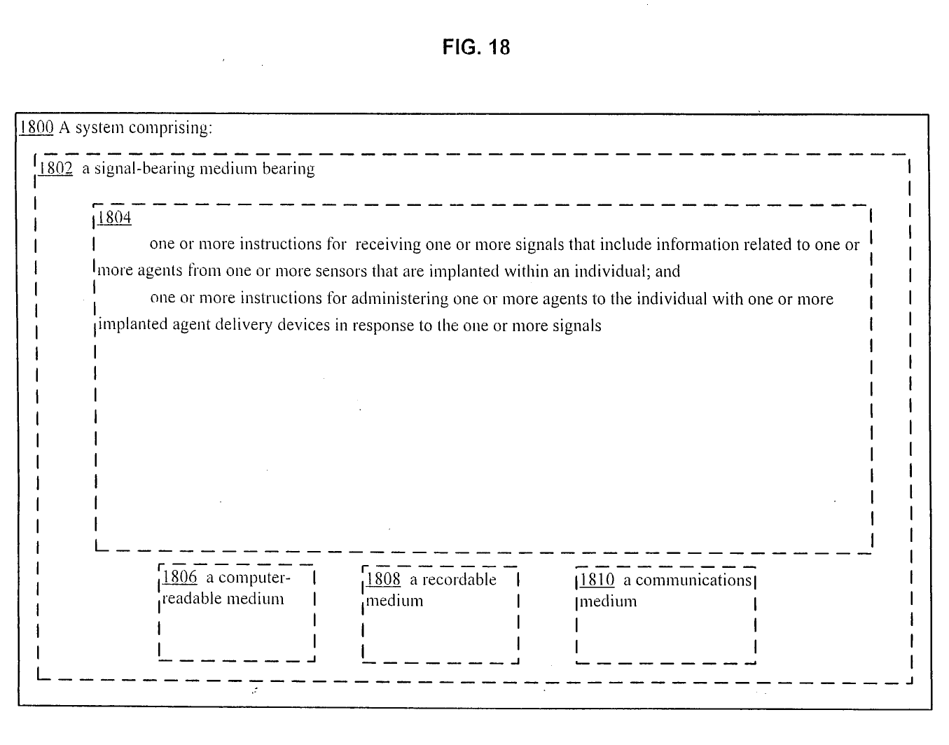

FIG. 18 illustrates a partial view of a system 1800 that includes a computer program 1804 for executing a computer process on a computing device. An embodiment of system 1800 is provided using a signal-bearing medium 1802 bearing one or more instructions for receiving one or more signals that include information related to one or more agents from one or more sensors that are implanted within an individual and one or more instructions for administering one or more agents to the individual with one or more implanted agent delivery devices in response to the one or more signals. The one or more instructions may be, for example, computer executable and/or logic-implemented instructions. In some embodiments, the signal-bearing medium 1802 may include a computer-readable medium 1806. In some embodiments, the signal-bearing medium 1802 may include a recordable medium 1808. In some embodiments, the signal-bearing medium 1802 may include a communications medium 1810.

FIG. 19A illustrates an embodiment of an agent delivery device 128 that includes a stepper motor 156 that is operably coupled to a moveable member 158 through a threaded member 1900. The agent delivery device 128 includes a device housing 140, an agent delivery control unit 146, one or more agents 162, and an agent permeable barrier 1902.

FIG. 19B illustrates an embodiment of an agent delivery device 128 that includes a squiggle motor 156 that is operably coupled to a moveable member 158 through a threaded member 1900. The agent delivery device 128 includes a device housing 140, an agent delivery control unit 146, one or more agents 162, and an agent permeable barrier 1902.

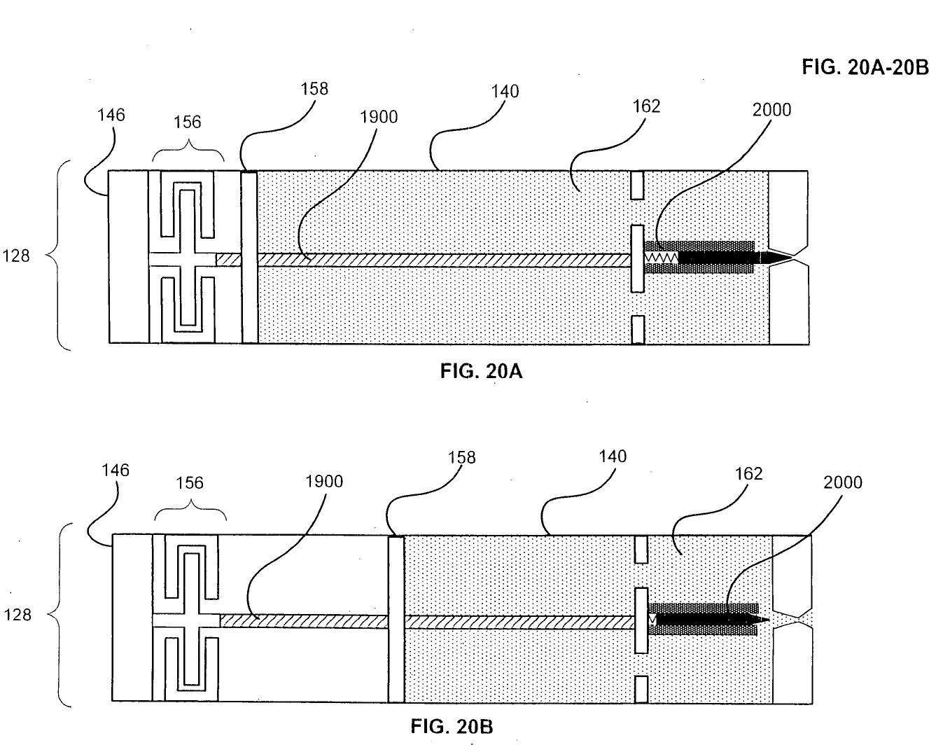

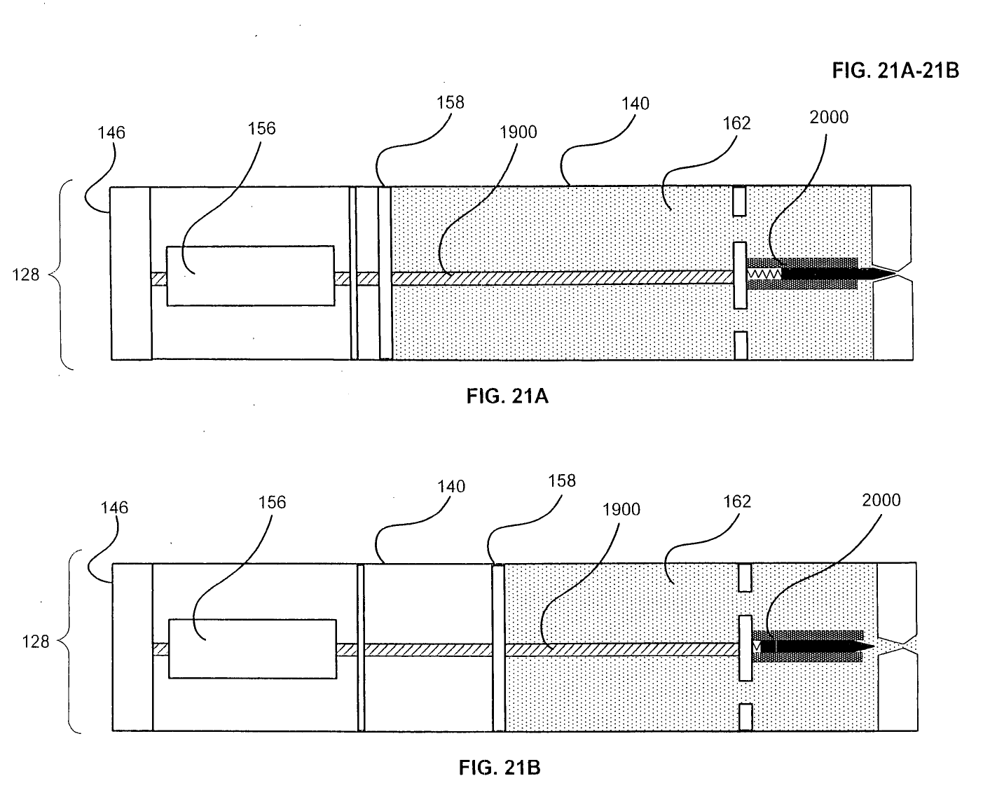

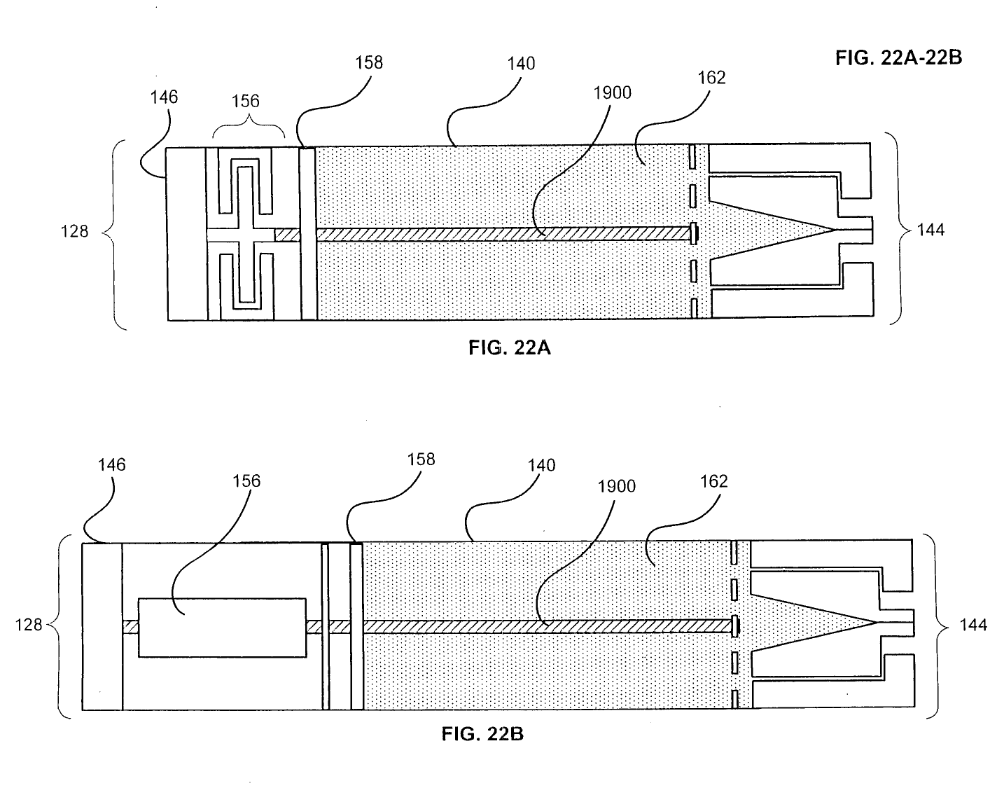

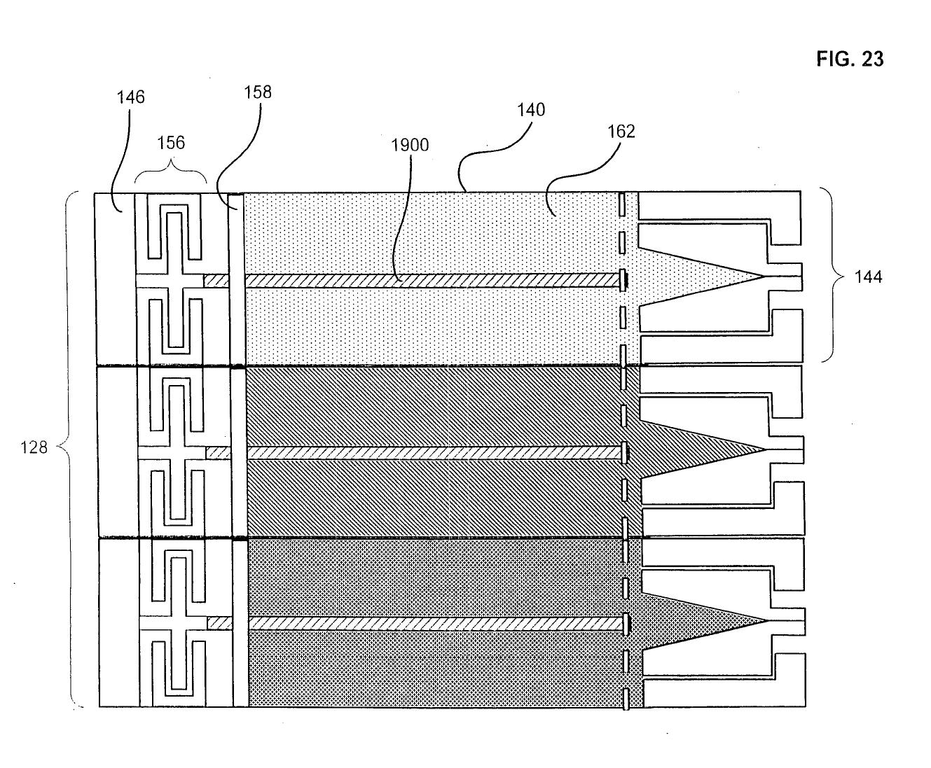

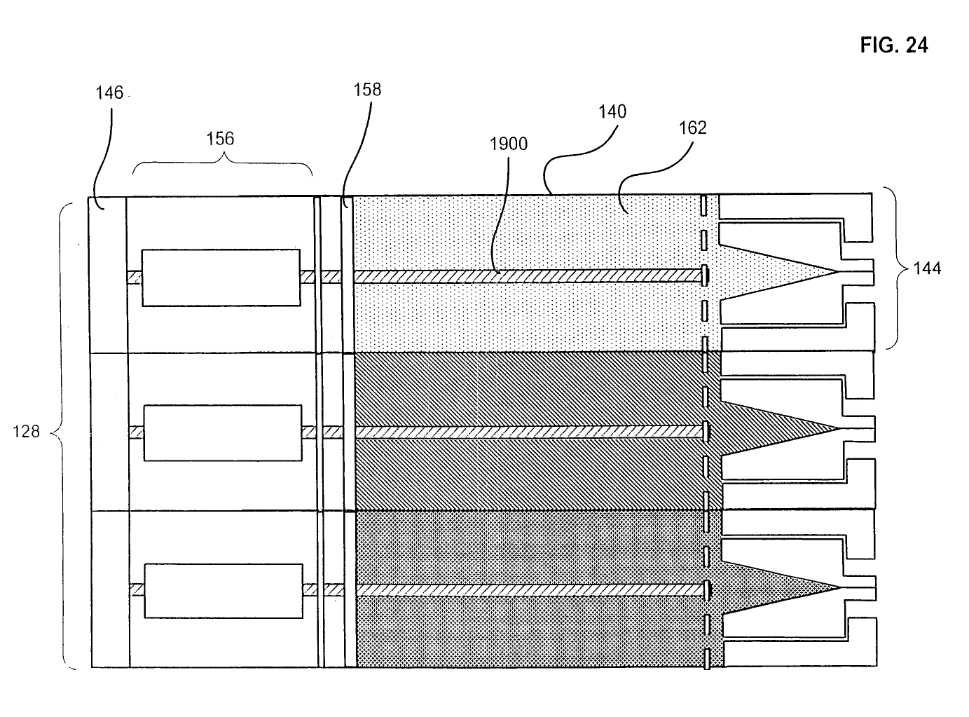

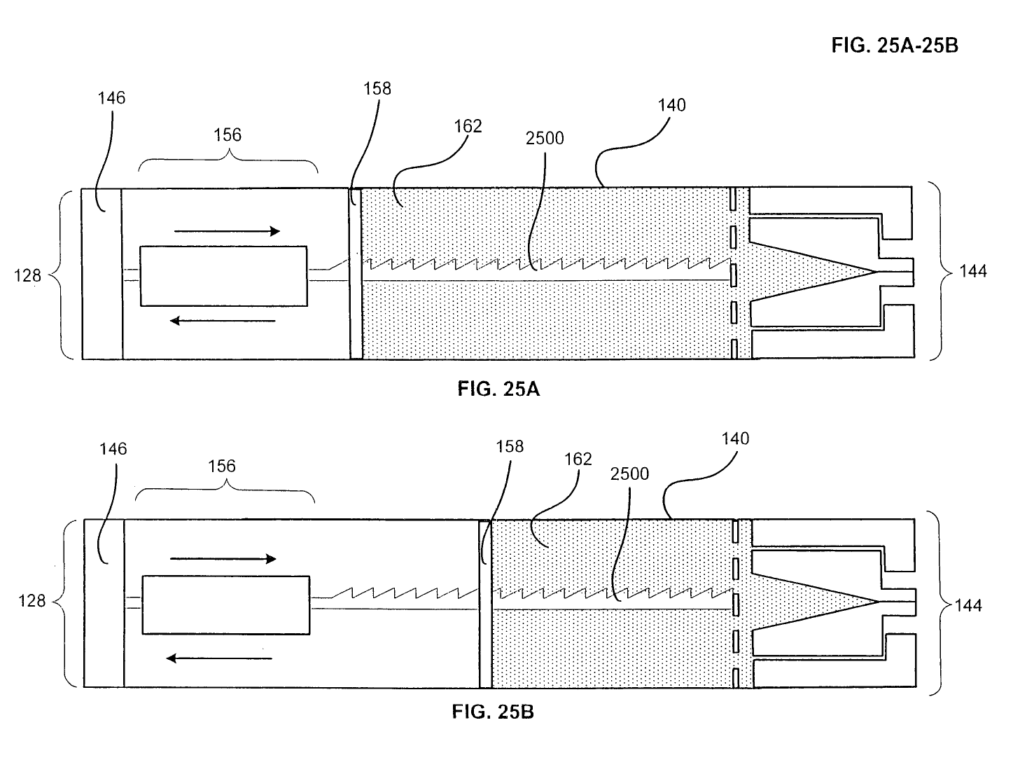

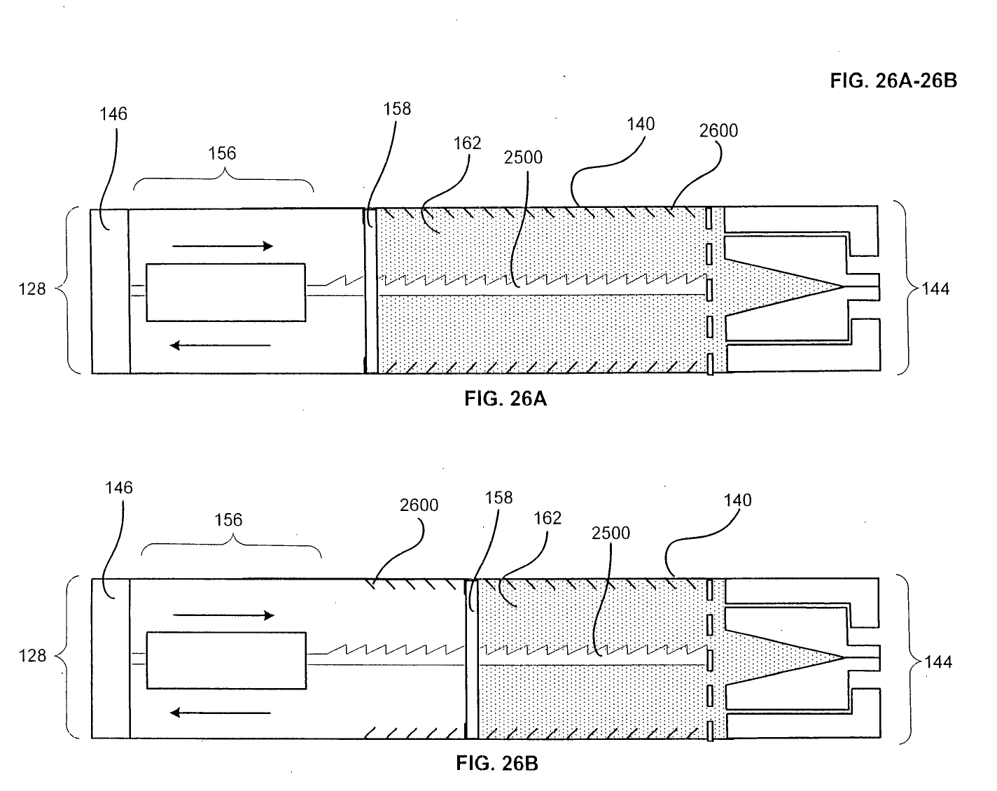

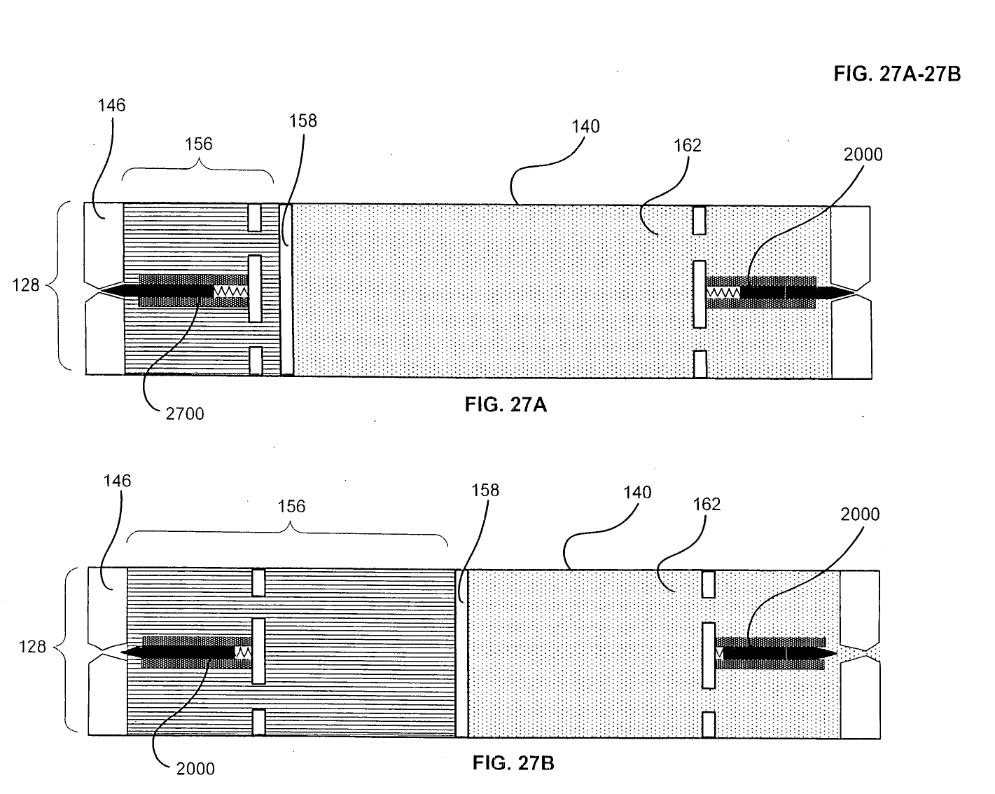

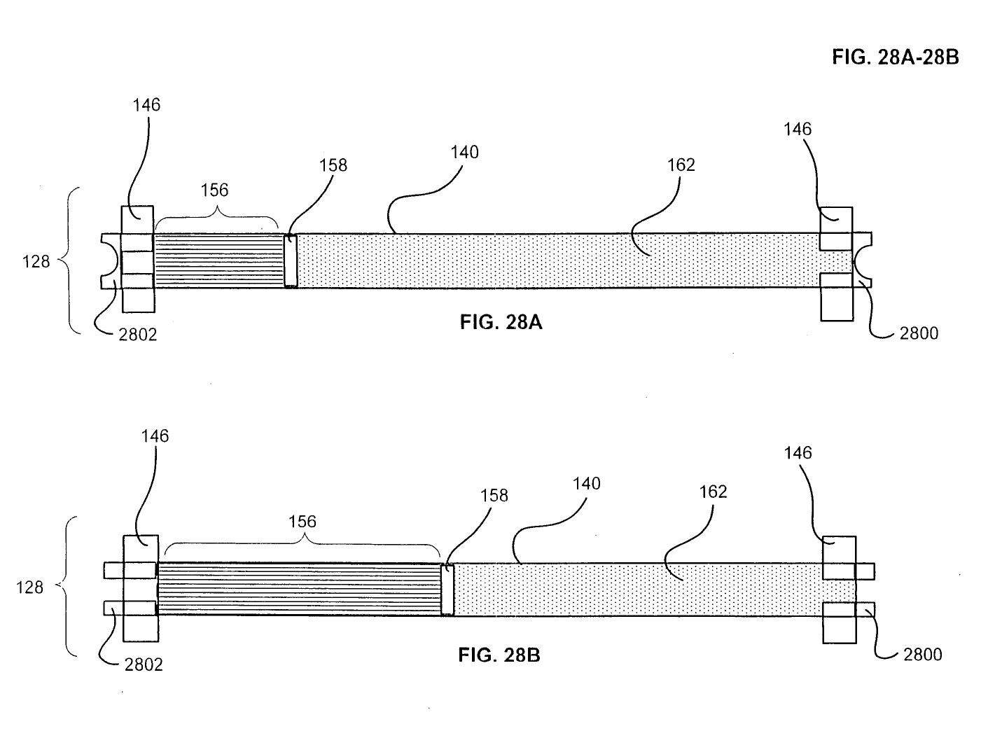

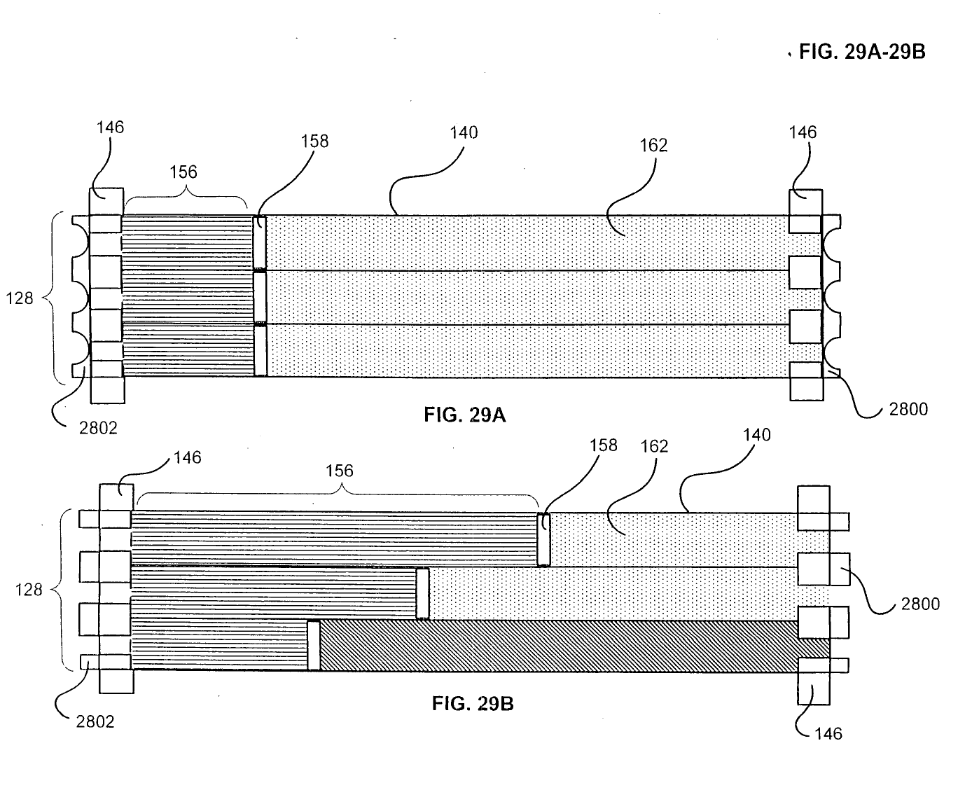

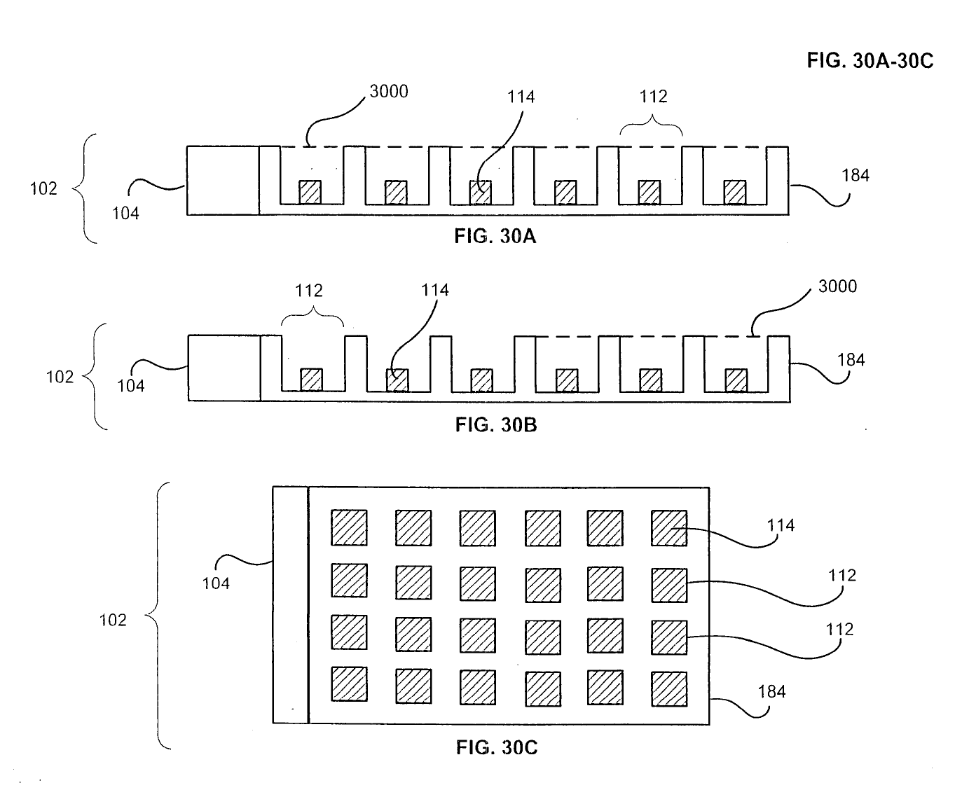

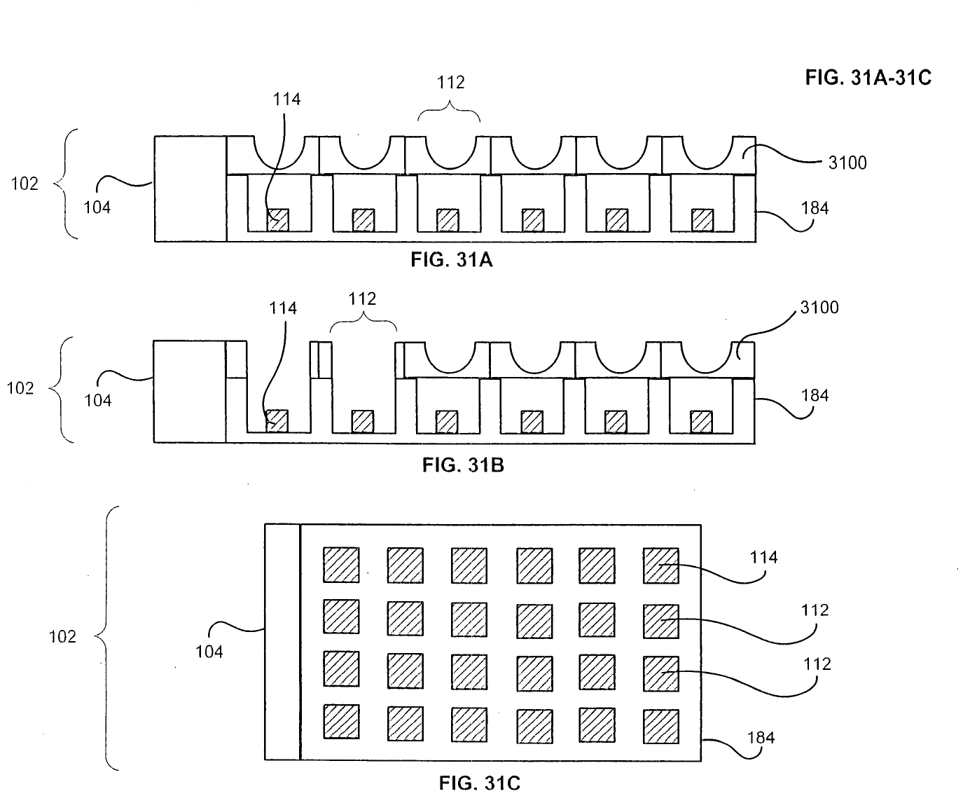

FIG. 20A illustrates an embodiment of an agent delivery device 128 that includes a stepper motor 156 that is operably coupled to a moveable member 158 through a threaded member 1900. The agent delivery device 128 includes a device housing 140, an agent delivery control unit 146, one or more agents 1662, and an electromagnetic exit port 2000 that is shown in the closed position.

FIG. 90B illustrates an embodiment of an agent delivery device 128 that includes a stepper motor 156 that is operably coupled to a moveable member 158 through a threaded member 1900. The agent delivery device 128 includes a device housing 140, an agent delivery control unit 146, one or more agents 162, and an electromagnetic exit port 2000 that is shown in the open position.

FIG. 21A illustrates an embodiment of an agent delivery device 128 that includes a squiggle motor 156 that is operably coupled to a moveable member 158 through a threaded member 1900. The agent delivery device 128 includes a device housing 140, an agent deliver) control unit 146, one or more agents 162, and an electromagnetic exit port 2000 that is shown in the closed position.

FIG. 21B illustrates an embodiment of an agent delivery device 128 that includes a squiggle motor 156 that is operably coupled to a moveable member 158 through a threaded member 1900. The agent delivery device 128 includes a device housing 140, an agent delivery control unit 146, one or more agents 162, and an electromagnetic exit port 2000 that is shown in the open position.

FIG. 22A illustrates an embodiment of an agent delivery device 128 that includes a stepper motor 156 that is operably coupled to a moveable member 158 through a threaded member 1900. The agent delivery device 128 includes a device housing 140, an agent delivery control unit 146, one or more agents 162, and a unidirectional exit port 144.

FIG. 22B illustrates an embodiment of an agent delivery device 128 that includes a squiggle motor 156 that is operably coupled to a moveable member 158 through a threaded member 1900. The agent delivery device 128 includes a device housing 140, an agent delivery control unit 146, one or more agents 162, and a unidirectional exit port 144.

FIG. 23 illustrates an embodiment of an agent delivery device 128 that includes an assembly of agent delivery devices 128. Each of the agent delivery devices 128 includes a stepper motor 156 that is operably coupled to a moveable member 158 through a threaded member 1900. Each of the agent delivery devices 128 includes a device housing 140, an agent delivery control unit 146, one or more agents 162, and a unidirectional exit port 144.

FIG. 24 illustrates an embodiment of an agent delivery device 128 that includes an assembly of agent delivery devices 128. Each of the agent delivery devices 128 includes a squiggle motor 156 that is operably coupled to a moveable member 158 through a threaded member 1900. Each of the agent delivery devices 128 includes a device housing 140, an agent delivery control unit 146, one or more agents 162, and a unidirectional exit port 144.

FIG. 25A illustrates an embodiment of an agent delivery device 128 that includes a piezoelectric linear motor 156 that is operably coupled to a moveable member 158 through a ratcheted member 2500. The agent delivery device 128 includes a device housing 140, an agent delivery control unit 146, one or more agents 162, and a unidirectional exit port 144.

FIG. 25B illustrates an embodiment of an agent delivery device 128 that includes a piezoelectric linear motor 156 that is operably coupled to a moveable member 158 through a ratcheted member 2500. The agent delivery device 128 includes a device housing 140, an agent delivery control unit 146, one or more agents 162, and a unidirectional exit port 144. The moveable member 158 is illustrated in an advanced position relative to the position of the moveable member 158 as illustrated in FIG. 25A.

FIG. 26A illustrates an embodiment of an agent delivery device 128 that includes a piezoelectric linear motor 156 that is operably coupled to a moveable member 158 through a ratcheted member 2500. The agent delivery device 128 includes a device housing 140, an agent delivery control unit 146, one or more agents 162, a unidirectional exit port 144, and moveable member retainers 2600.

FIG. 26B illustrates an embodiment of an agent delivery device 128 that includes a piezoelectric linear motor 156 that is operably coupled to a moveable member 158 through a ratcheted member 2500. The agent delivery device 128 includes a device housing 140, an agent deliver, control unit 146, one or more agents 162, a unidirectional exit port 144, and moveable member retainers 2600. The moveable member 158 is illustrated in an advanced position relative to the position of the moveable member 158 as illustrated in FIG. 26A.

FIG. 27A illustrates an embodiment of an agent delivery device 128 that includes an osmotic motor 156 that facilitates movement of a moveable member 158 through introduction of solute into the osmotic motor 156. The agent delivery device 128 includes a device housing 140, an agent delivery control unit 146, one or more agents 162, an electromagnetic exit port 2000 that is shown in the closed position, and an electromagnetic entry port 2700 that is shown in the closed position.

FIG. 27B illustrates an embodiment of an agent delivery device 128 that includes an osmotic motor 156 that facilitates movement of a moveable member 158 through introduction of solute into the osmotic motor 156. The agent delivery device 128 includes a device housing 140, an agent delivery control unit 146, one or more agents 162, an electromagnetic exit port 2000 that is shown in the open position, and an electromagnetic entry port 2700 that is shown in the open position. The moveable member 158 is illustrated in an advanced position relative to the position of the moveable member 158 as illustrated in FIG. 27A.

FIG. 28A illustrates an embodiment of an agent delivery device 128 that includes an osmotic motor 156 that facilitates movement of a moveable member 158 through introduction of solute into the osmotic motor 156. The agent delivery device 128 includes a device housing 140, agent delivery control units 146, one or more agents 162, an exit port 2800 made from a shape memory material that is shown in the closed position, and an entry port 2802 made from a shape memory material that is shown in the closed position.

FIG. 28B illustrates an embodiment of an agent delivery device 128 that includes an osmotic motor 156 that facilitates movement of a moveable member 158 through introduction of solute into the osmotic motor 156. The agent delivery device 128 includes a device housing 140, agent delivery control units 146, one or more agents 162, an exit port 2800 made from a shape memory material that is shown in the open position, and an entry port 2802 made from a shape memory material that is shown in the open position. The moveable member 158 is illustrated in an advanced position relative to the position of the moveable member 158 as illustrated in FIG. 28A.

FIG. 29A illustrates an embodiment of an agent delivery device 128 that includes an assembly of individual agent delivery devices 128. Each agent delivery device 128 includes an osmotic motor 156 that facilitates movement of a moveable member 158 through introduction of solute into the osmotic motor 156. Each agent delivery device 128 includes a device housing 140, agent delivery control units 146, one or more agents 162, an exit port 2800 made from a shape memory material that is shown in the closed position, and an entry port 2802 made from a shape memory material that is shown in the closed position.

FIG. 29B illustrates an embodiment of an agent delivery device 128 that includes an assembly of individual agent delivery devices 128. Each agent delivery device 128 includes an osmotic motor 156 that facilitates movement of a moveable member 158 through introduction of solute into the osmotic motor 156. Each agent delivery device 128 includes a device housing 140, agent delivery control units 146, one or more agents 162, an exit port 2800 made from a shape memory material that is shown in the open position, and an entry port 9802 made from a shape memory material that is shown in the open position. The moveable members 158 are shown in an advanced position relative to their position as illustrated in FIG. 29A.

FIG. 30A illustrates a side-view of an embodiment of sensor 102 that includes a sensor control unit 104, and a sensor housing 184 that includes selectively accessible portions 112 that are covered with a sacrificial layer 3000 and which enclose sensor detectors 114. All of the selectively accessible portions 112 are shown as being sequestered from the outside environment.

FIG. 30B illustrates a side-view of an embodiment of sensor 102 that includes a sensor control unit 104, and a sensor housing 184 that includes selectively accessible portions 112 that are covered with a sacrificial layer 3000 and which enclose sensor detectors 114. The sacrificial layer 3000 is shown as having been removed from three of the selectively accessible portions 112 of the sensor 102 to expose three sensor detectors 114 to the outside environment.

FIG. 30C illustrates a top-view of an embodiment of sensor 102 that includes a sensor control unit 104, and a sensor housing 184 that includes selectively accessible portions 112 that enclose sensor detectors 114.

FIG. 31A illustrates a side-view of an embodiment of sensor 102 that includes a sensor control unit 104, and a sensor housing 184 that includes selectively accessible portions 112 that are covered with a shape memory material 3100 and which enclose sensor detectors 114. All of the selectively accessible portions 11′ are shown as being sequestered from the outside environment.

FIG. 31B illustrates a side-view of an embodiment of sensor 102 that includes a sensor control unit 104, and a sensor housing 184 that includes selectively accessible portions 112 that are covered with a shape memory material 3100 and which enclose sensor detectors 114. The shape memory material 3100 covering two of the selectively accessible portions 19 is shown as having been reshaped to expose two sensor detectors 114 to the outside environment.

FIG. 31C illustrates a top-view of an embodiment of sensor 102 that includes a sensor control unit 104, and a sensor housing 184 that includes selectively accessible portions 119 and which enclose sensor detectors 114.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, anti of which may vary. Those skilled in the art will recognize that optical aspects of implementations will typically employ optically-oriented hardware, software, and or firmware.

In some implementations described herein, logic and similar implementations may include software or other control structures suitable to operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In a general sense, those skilled in the alt will recognize that the various embodiments described herein can be implemented, individually and/or collectively, by various types of electro-mechanical systems having a wide range of electrical components such as hardware, software, firmware, and/or virtually any combination thereof, and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or an), non-electrical analog thereto, such as optical or other analogs. Those skilled in the art will also appreciate that examples of electromechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or deices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will recognize that at least a portion of the devices and/or processes described herein can be integrated into an image processing system. Those having skill in the art will recognize that a typical image processing system generally includes one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system may be implemented utilizing suitable commercially available components, such as those typically found in digital still systems and/or digital motion systems.

Those skilled in the art still recognize that at least a portion of the devices and/or processes described herein can be integrated into a data processing system. Those having skill in the art will recognize that a data processing system generally includes one or more of a system unit housing, a video displays device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems. Those skilled in the art will recognize that at least a portion of the devices and/or processes described herein can be integrated into a mote system. Those having skill in the art will recognize that a typical mote system generally includes one or more memories such as volatile or non-volatile memories, processors such as microprocessors or digital signal processors, computational entities such as operating systems, user interfaces, drivers, sensors, actuators, applications programs, one or more interaction devices (e.g., an antenna USB ports, acoustic ports, etc.), control systems including feedback loops and control motors (e.g., feedback for sensing or estimating position and/or velocity; control motors for moving and/or adjusting components and/or quantities). A mote system may be implemented utilizing suitable components, such as those found in mote computing/communication systems. Specific examples of such components entail such as Intel Corporation's and/or Crossbow Corporation's mote components and supporting hardware, software, and/or firmware.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.). (e) a communications system (e.g., a networked system, a telephone system, a Voice over IP system, etc.), (f) a business entity (e.g., an Internet Service Provider (ISP) entity such as Comcast Cable, Quest, Southwestern Bell. etc.), or (g) a wired/wireless services entity (e.g. Sprint, Cingular, Nextel, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory). A sale of a system or method may likewise occur in a territory even if components of the system or method are located and/or used outside the territory. Further, implementation of at least part of a system for performing a method in one territory does not preclude use of the system in another territory.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Those skilled in the art will appreciate that a user may be representative of a human user, a robotic user (e.g., computational entity), and/or substantially any combination thereof (e.g., a user may be assisted by one or more robotic agents) unless context dictates otherwise.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity. The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless contest requires otherwise. While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations mass be performed in other orders than those which are illustrated, or mats be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

1-46. (canceled)

47. A system comprising:
circuitry for receiving one or more signals that include information related to one or more agents from one or more sensors that are implanted within an individual; and
circuitry for administering one or more agents to the individual with one or more agent delivery devices that are implanted within the individual and responsive to the circuitry for receiving the one or more signals.

48. The system of claim 47, wherein the circuitry for receiving one or more signals that include information related to one or more agents from one or more sensors that are implanted within an individual comprises:
circuitry for receiving the one or more signals directly from the one or more sensors.

49. The system of claim 47, wherein the circuitry for receiving one or more signals that include information related to one or more agents from one or more sensors that are implanted within an individual comprises:
circuitry for receiving the one or more signals from one or more implanted relay devices.

50. The system of claim 47, wherein the circuitry for receiving one or more signals that include information related to one or more agents from one or more sensors that are implanted within an individual comprises:
circuitry for receiving the one or more signals that include one or more ultrasonic signals.

51. The system of claim 47, wherein the circuitry for receiving one or more signals that include information related to one or more agents from one or more sensors that are implanted within an individual comprises:
circuitry for receiving the one or more signals that include one or more infrared signals.

52. The system of claim 47, wherein the circuitry for receiving one or more signals that include information related to one or more agents from one or more sensors that are implanted within an individual comprises:
circuitry for receiving the one or more signals that include one or more acoustic signals.

53. The system of claim 47, wherein the circuitry for receiving one or more signals that include information related to one or more agents from one or more sensors that are implanted within an individual comprises:
circuitry for receiving the one or more signals that include one or more optical signals.

54. The system of claim 47, wherein the circuitry for receiving one or more signals that include information related to one or more agents from one or more sensors that are implanted within an individual comprises:
circuitry for receiving the one or more signals that include one or more electromagnetic signals.

55. The system of claim 47, wherein the circuitry for receiving one or more signals that include information related to one or more agents from one or more sensors that are implanted within an individual comprises:
circuitry for receiving the one or more signals that include one or more microwave signals.

56. The system of claim 47, wherein the circuitry for receiving one or more signals that include information related to one or more agents from one or more sensors that are implanted within an individual comprises:
circuitry for receiving the one or more signals that include one or more radio signals.

57. The system of claim 47, wherein the circuitry for receiving one or more signals that include information related to one or more agents from one or more sensors that are implanted within an individual comprises:
circuitry for receiving the one or more signals that include one or more radio frequency signals.

58. The system of claim 47, wherein the circuitry for receiving one or more signals that include information related to one or more agents from one or more sensors that are implanted within an individual comprises:
circuitry for receiving the one or more signals substantially continuously from the one or more sensors

59. The system of claim 47, wherein the circuitry for receiving one or more signals that include information related to one or more agents from one or more sensors that are implanted within an individual comprises:
circuitry for receiving the one or more signals from the one or more sensors that are configured to present one or more detectors at different times.

60. The system of claim 47, wherein the circuitry for receiving one or more signals that include information related to one or more agents from one or more sensors that are implanted within an individual comprises:
circuitry for receiving the one or more signals from the one or more sensors that are configured to present one or more detectors at different times through use of a sacrificial layer.

61. The system of claim 47, wherein the circuitry for receiving one or more signals that include information related to one or more agents from one or more sensors that are implanted within an individual comprises:
circuitry for receiving the one or more signals from the one or more sensors that are configured to present one or more detectors at different times through use of one or more shape memory polymers.

62. The system of claim 47, wherein the circuitry for receiving one or more signals that include information related to one or more agents from one or more sensors that are implanted within an individual comprises:
circuitry for receiving the one or more signals from the one or more sensors according to one or more time schedules.

63. The system of claim 47, wherein the circuitry for receiving one or more signals that include information related to one or more agents from one or more sensors that are implanted within an individual comprises:
circuitry for receiving the one or more signals from the one or more sensors in response to one or more queries.

64. The system of claim 47, wherein the circuitry for receiving one or more signals that include information related to one or more agents from one or more sensors that are implanted within an individual comprises:
circuitry for receiving the one or more signals that include information related to one or more concentrations of the one or more agents within the individual.

65. The system of claim 47, wherein the circuitry for receiving one or more signals that include information related to one or more agents from one or more sensors that are implanted within an individual comprises:
circuitry for receiving the one or more signals that include information related to one or more concentrations of one or more metabolites of the one or more agents within the individual.

66. (canceled)

67. The system of claim 47, wherein the circuitry for receiving one or more signals that include information related to one or more agents from one or more sensors that are implanted within an individual comprises:
circuitry for receiving the one or more signals that include information related to one or more concentrations of nitric oxide within the individual.

68. The system of claim 47, wherein the circuitry for receiving one or more signals that include information related to one or more agents from one or more sensors that are implanted within an individual comprises:
circuitry for receiving the one or more signals that include information related to one or more agents that include one or more pharmaceutical agents.

69. (canceled)

70. The system of claim 47, wherein the circuitry for receiving one or more signals that include information related to one or more agents from one or more sensors that are implanted within an individual comprises:
circuitry for receiving the one or more signals that include information related to one or more agents that include one or more hormones.

71. (canceled)

72. The system of claim 47, wherein the circuitry for receiving one or more signals that include information related to one or more agents from one or more sensors that are implanted within an individual comprises:
circuitry for receiving the one or more signals that include information related to one or more agents that include one or more cytokines.

73. (canceled)

74. The system of claim 47, wherein the circuitry for administering one or more agents to the individual with one or more agent delivery devices that are implanted within the individual and responsive to the circuitry for receiving the one or more signals comprises:
circuitry for maintaining the one or more agents substantially within one or more concentration ranges within the individual.

75. The system of claim 47, wherein the circuitry for administering one or more agents to the individual with one or more agent delivery devices that are implanted within the individual and responsive to the circuitry for receiving the one or more signals comprises:
circuitry for maintaining the one or more agents substantially at one or more setpoints within the individual.

76. The system of claim 47, wherein the circuitry for administering one or more agents to the individual with one or more agent delivery devices that are implanted within the individual and responsive to the circuitry for receiving the one or more signals comprises:
circuitry for administering one or more pharmaceutical agents.

77. (canceled)

78. The system of claim 47, wherein the circuitry for administering one or more agents to the individual with one or more agent delivery devices that are implanted within the individual and responsive to the circuitry for receiving the one or more signals comprises:
circuitry for administering one or more hormones.

79. (canceled)

80. The system of claim 47, wherein the circuitry for administering one or more agents to the individual with one or more agent delivery devices that are implanted within the individual and responsive to the circuitry for receiving the one or more signals comprises:
circuitry for administering one or more cytokines.

81. (canceled)

82. The system of claim 47, wherein the circuitry for administering one or more agents to the individual with one or more agent delivery devices that are implanted within the individual and responsive to the circuitry for receiving the one or more signals comprises:
circuitry for administering one or more nitric oxide donors to the individual.

83. The system of claim 47, wherein the circuitry for administering one or more agents to the individual with one or more agent delivery devices that are implanted within the individual and responsive to the circuitry for receiving the one or more signals comprises:
circuitry for administering one or more photolyzable nitric oxide donors to the individual.

84. The system of claim 47, wherein the circuitry for administering one or more agents to the individual with one or more agent delivery devices that are implanted within the individual and responsive to the circuitry for receiving the one or more signals comprises:
circuitry for administering one or more nitric oxide donors to the individual that include one or more diazeniumdiolates, trans-[RuCl([15]aneN4)NO]+2, nitrosyl ligands, 6-Nitrobenzo[a]pyrene, S-nitroso-glutathione, S-nitrosothiols, 2-Methyl-2-nitrosopropane, nitroglycerine, L-arginine, or imidazolyl derivatives.

85. The system of claim 47, wherein the circuitry for administering one or more agents to the individual with one or more agent delivery devices that are implanted within the individual and responsive to the circuitry for receiving the one or more signals comprises:
circuitry for administering the one or more agents with one or more osmotic agent delivery devices.

86. The system of claim 47, wherein the circuitry for administering one or more agents to the individual with one or more agent delivery devices that are implanted within the individual and responsive to the circuitry for receiving the one or more signals comprises:
circuitry for administering the one or more agents with one or more motorized agent delivery devices.

87. The system of claim 47, wherein the circuitry for administering one or more agents to the individual with one or more agent delivery devices that are implanted within the individual and responsive to the circuitry for receiving the one or more signals comprises:
circuitry for administering the one or more agents with one or more charge activated agent delivery devices.

88. The system of claim 47, wherein the circuitry for administering one or more agents to the individual with one or more agent delivery devices that are implanted within the individual and responsive to the circuitry for receiving the one or more signals comprises:
circuitry for transmitting the one or more signals in response to administration of the one or more agents to the individual.

89. (canceled)

90. A system comprising:
a signal-bearing medium bearing:
one or more instructions for receiving one or more signals that include information related to one or more agents from one or more sensors that are implanted within an individual; and
one or more instructions for administering one or more agents to the individual with one or more implanted agent delivery devices in response to the one or more signals.

91. The system of claim 90, wherein the signal-bearing medium includes a computer-readable medium.

92. The system of claim 90, wherein the signal-bearing medium includes a recordable medium.

93. The system of claim 90, wherein the signal-bearing medium includes a communications medium.

94. A system comprising:
circuitry for receiving one or more acoustic signals that include information related to one or more agents from one or more sensors that are implanted within an individual; and
circuitry for administering one or more agents to the individual with one or more implanted agent delivery devices that maintain the one or more agents substantially at one or more setpoints within the individual in response to the circuitry for receiving one or more acoustic signals.
